SEQUENCE LISTING (3 sequences)

SEQ ID NO: 1 - protein, 13 aa, Artificial Sequence
  Synthetic construct
PXCVXXXRCXGCC

SEQ ID NO: 2 - DNA, 30 bp, Artificial Sequence
  Primer
cacggatcccacaggatctagttcaggttc

SEQ ID NO: 3 - DNA, 50 bp, Artificial Sequence
  Primer
gcgaattcttagtgatggtgatggtgatgcagattagacttgtccgaggt

(19) United States
(12) Patent Application Publication
(10) Pub. No.: US 2011/0318365 A1
(43) Pub. Date: Dec. 29, 2011

(54) METHODS FOR TREATMENT OF DEGENERATIVE DISEASE ASSOCIATED WITH APOPTOSIS

(75) Inventor: Xuri Li, Bethesda, MD (US)

(73) Assignee: The United States of America, as represented by the Secretary, Department of health and Human, Bethesda, MD (US)

(21) Appl. No.: 12/674,331

(22) PCT Filed: Sep. 11, 2008
(86) PCT No.: PCT/US08/75988

§ 371 Date: Aug. 11, 2010

Related U.S. Application Data
(60) Provisional application No. 60/972,780, filed on Sep. 15, 2007.

(51) Int. Cl. A61K 39/395; A61K 31/7088; C40B 30/04; C12Q 1/68; C12Q 1/02; A61P 27/16; A61P 25/00; A61P 27/02; A61P 27/06; A61P 21/00; A61P 25/28; A61P 25/16; A61K 38/18; G01N 33/566

(52) U.S. Cl. 424/172.1; 514/8.1; 514/44 R; 506/9; 435/6.12; 435/29; 435/7.21; 435/6.1

(57) ABSTRACT

A method for treating a degenerative disease or neurodegenerative disease in a mammalian subject is provided. The method provides administering VEGF-B polypeptide or functional variant or mimetic thereof in an amount effective to reduce or eliminate the degenerative disease in the mammalian subject without having any substantial angiogenic effect across all dosage levels.

### CROSS REFERENCE TO RELATED APPLICATIONS

This application claims priority to U.S. Application No. 60/972,780 filed Sep. 15, 2007, the entire disclosure of which is incorporated herein by reference.

### FIELD OF THE INVENTION

The invention generally relates to methods for treating a degenerative disease, neurodegenerative disease, ischemic disease, or a disease associated with apoptosis-mediated cell death in a mammalian subject. The method provides administering VEGF-B polypeptide or functional variant or mimetic thereof in an amount effective to reduce or eliminate the degenerative disease in the mammalian subject.

### BACKGROUND OF THE INVENTION

The vascular endothelial growth factor (VEGF) family incorporates five structurally related ligands that bind differentially to three receptor tyrosine kinases (VEGFR-1, -2, and -3) and the semaphorin receptors neuropilin (NP-1 and -2). Among the five VEGF family members, VEGF is the prototype angiogenic factor with a potent and “universal” angiogenic effect in most physiological and pathological conditions (1-3). Placenta growth factor (P1GF) is believed to play important roles in pathological angiogenesis (4), while VEGF-C and VEGF-D are mainly involved in lymphangiogenesis (5). VEGF has recently been shown to be a potent neuroprotective factor (6, 7). However, the therapeutic potential of its neuroprotective effect is limited by its potent and general activity in inducing undesired angiogenesis and blood vessel leakage, which is highly detrimental in most conditions.

Compared with the other members of the VEGF family, VEGF-B has received much less attention, and its biological function remains debatable (8, 9). VEGF-B shares a high degree of sequence homology with VEGF and P1GF. It binds to the tyrosine kinase receptor VEGFR-1 and its co-receptor neuropilin-1 (10, 11). VEGF-B is highly expressed in most tissues and organs (12, 13). VEGF-B in most conditions seems to be “redundant” or “inert.” In contrast to VEGF deficient mice (14, 15), VEGF-B deficient mice are healthy and fertile, with apparently normal developmental and physiological angiogenesis (16, 17). In contrast to VEGF and P1GF, VEGF-B is not required for the neovessel formation in the proliferative retinopathy (18) and blood vessel remodeling in pulmonary hypertension (19). Adenoviral VEGF-B gene transfer in the hind limb (20) and carotid artery (21) did not induce any angiogenic response, while the other VEGF family members did. Moreover, numerous studies have shown that VEGF-B has no effect on blood vessel permeability (19, 22-24), in contrast to the other VEGF family members.

Current functional analysis of VEGF-B has led to inconsistent and controversial results. While one study reported smaller heart and abnormal coronary artery vasculature caused by VEGF-B deficiency in mice (17), this was not observed by another independent study (16). Several reports have shown an angiogenic effect of VEGF-B (24-26), whereas other studies have observed the lack of angiogenic activity of VEGF-B in different model systems (16, 18, 20, 21, 27). VEGF-B was reported to play a role in pulmonary hypertension (28), however, this was not observed in another independent study (19). In summary, due to various reasons including distinct gene-targeting approaches used, different genetic background of the animals in different studies, and experimental variations among different research groups, the reports published thus far with regard to the function of VEGF-B have been inconsistent. A need exists in the art to develop effective therapeutic compounds for treatment of a degenerative disease, e.g., a neurodegenerative disease; in particular, a treatment that provides therapeutic biological compounds that can reduce or eliminate the neurodegenerative disease without stimulating angiogenesis in a mammalian subject.

### SUMMARY OF THE INVENTION

The invention generally relates to methods for treating a degenerative disease a disease associated with apoptosis-mediated cell death in a mammalian subject. The method provides administering VEGF-B polypeptide or functional variant or mimetic thereof in an amount effective to reduce or eliminate the degenerative disease in the mammalian subject without having any substantial angiogenic effect across all dosage levels. The invention further relates to methods for treating pathological conditions associated with excessive apoptosis in a mammalian subject without having any substantial angiogenic effect across all dosage levels. The method provides administering VEGF-B polypeptide, or functional variant or mimetic thereof, in an amount effective to reduce or eliminate the diseased conditions in the mammalian subject without having any substantial angiogenic effect across all dosage levels. The disease can be, for example, a neurodegenerative disease or ischemic disease.

A method for treating a degenerative disease in a mammalian subject is provided which comprises administering a VEGF-B polypeptide, or functional variant or mimetic thereof, in an amount effective to reduce or eliminate the degenerative disease in the mammalian subject without having any substantial angiogenic effect across all dosage levels. The disease includes, but is not limited to, a neurodegenerative disease, neurodegenerative ocular disease, neurovascular degenerative disease, or combinations thereof. In one aspect, the disease is a condition involving apoptosis-mediated-cell death. The VEGF-B polypeptide, or functional variant or mimetic, can include, but is not limited to, a VEGF-B polypeptide fragment, peptide mimetic, nucleic acid, RNA, DNA, small chemical molecule, or antibody. The VEGF-B polypeptide functional variant or mimetic can be a conservative amino acid substitution or peptide mimetic substitution.

A method for inhibiting apoptosis in a cell or tissue of a mammalian subject which comprises administering VEGF-B polypeptide or functional variant or mimetic thereof in an amount effective to reduce or eliminate apoptosis in the cell or tissue of the mammalian subject without having any substantial angiogenic effect across all dosage levels. In one aspect, apoptosis occurs in a neural tissue, for example, in brain or retina. In a further aspect, apoptosis occurs in endothelial cells, pericytes, Muller glial cells, neurons, nerves, cardiac myocytes, or myofibers. In a further aspect, inhibition of apoptosis in the cell or tissue occurs without stimulating angiogenesis. The disease includes, but is not limited to, a neurodegenerative disease, neurodegenerative ocular disease, neurovascular degenerative disease, or ischemic disease. In one aspect, the disease is a condition involving apoptosis-mediated-cell death. The VEGF-B polypeptide functional variant or mimetic can include, but is not limited to, a VEGF-B polypeptide fragment, peptide mimetic, nucleic acid, RNA, DNA, small chemical molecule, or antibody. The VEGF-B polypeptide functional variant or mimetic can be a conservative amino acid substitution or peptide mimetic substitution.

A method for identifying a compound which inhibits apoptosis in a cell or tissue without having any substantial angiogenic effect across all dosage levels is provided which comprises contacting a VEGF-B polypeptide functional variant or mimetic test compound with a cell-based assay system comprising a cell expressing VEGFR-1 or neuropilin-1 and apoptosis/cell death related genes, and detecting an effect of the test compound on VEGFR-1- or neuropilin-1-signaling and on inhibition of apoptosis/cell death related gene expression, effectiveness of the test compound in the assay being indicative of inhibition of apoptosis activity in the cell or tissue. The method further comprises comparing the effect of the test compound to an effect of VEGF-B polypeptide on inhibition of apoptosis/cell death related gene expression. The compound can include, but is not limited to, a VEGF-B polypeptide functional variant or mimetic is a VEGF-B polypeptide fragment, VEGF-B peptide mimetic, small chemical molecule, nucleic acid, RNA, DNA, or antibody. In one aspect, apoptosis/cell death related gene expression is BH3-only protein gene expression In a detailed aspect, the BH3-only protein gene expression is expression of genes Bmf, Hrk, Puma, Noxa (Pmaip1), Bad, Bid, or Bik. In a further aspect, apoptosis/cell death related gene expression is expression of genes TrP53inp1 or DCN.

### BRIEF DESCRIPTION OF THE DRAWINGS

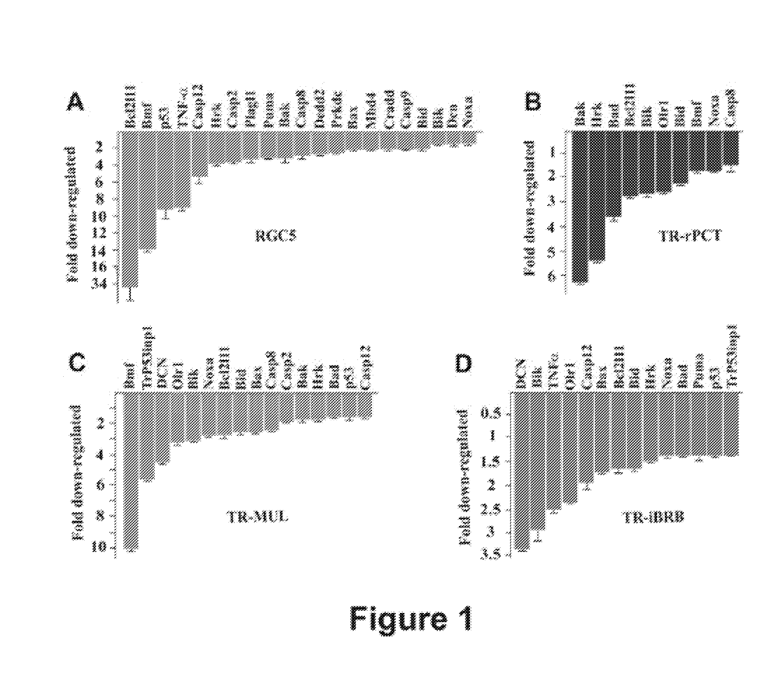

FIG. 1 shows VEGF-B₁₆₇ inhibits the expression of the BH3-only protein and other apoptotic/cell death-related genes in multiple cell lines.
(A) VEGF-B₁₆₇ treatment inhibited the expression of the BH3-only protein (Bmf, Hrk, Puma, Bid, Bik, Noxa) and other apoptotic/cell death-related genes in the rat retinal ganglion cell-derived cell line RGC5 using real-time PCR assay.
(B) VEGF-B₁₆₇ treatment inhibited the expression of the BH3-only protein (Hrk, Bik, Bid, Bmf, Noxa) and other apoptotic/cell death-related genes in the immortalized rat retinal pericyte cell line TR-rPCT.
(C) VEGF-B₁₆₇ treatment inhibited the expression of the BH3-only protein (Bmf, Bik, Noxa, Bid, Hrk) and other apoptotic/cell death-related genes in the immortalized rat retinal Müller cell line TR-MUL.
(D) VEGF-B₁₆₇ treatment inhibited the expression of the BH3-only protein (Bik, Bid, Hrk, Noxa, Puma) and other apoptotic/cell death-related genes in the immortalized rat retinal endothelial cell line TR-iBRB.

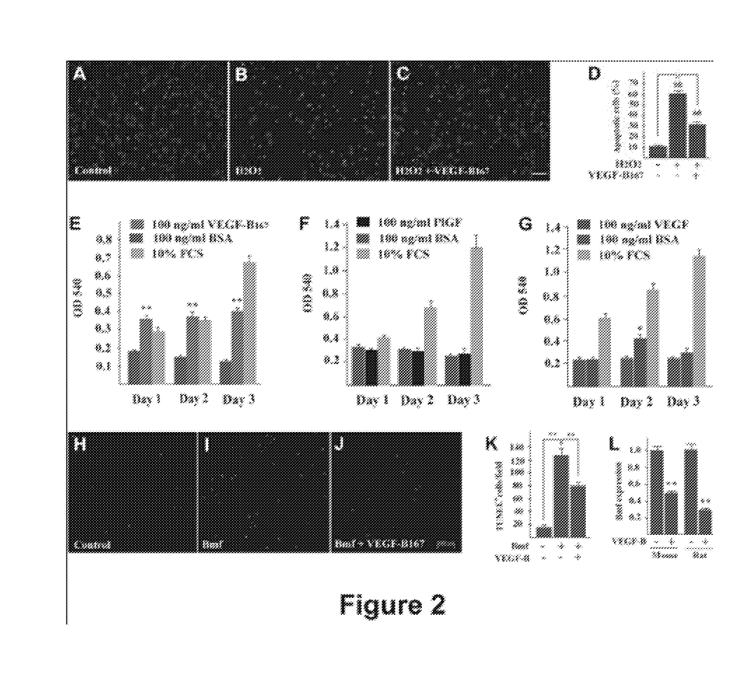

FIG. 2 shows survival effect of VEGF-B on the RGC5 cells
(A, B) Hydrogen peroxide (H₂O₂) treatment led to oxidative stress-induced apoptosis in the RGC5 cells using the terminal deoxynucleotidyl transferase dUTP nick end labeling (TUNEL) assay.
(C, D) VEGF-B₁₆₇ treatment inhibited the H₂O₂-induced apoptosis. Scale bar: 50 μm
(E) VEGF-B rescued serum deprivation-induced cell death in the RGC5 cells at different time point.
(F) P1GF did not rescue serum deprivation-induced cell death in the RGC5 cells at different time point.
(G) VEGF had a weak survival effect on the RGC5 cells. BSA: bovine serum albumin; FCS: fetal calf serum.
(H, I) Bmf overexpression in the RGC5 cells led to apoptosis using the TUNEL assay.
(J, K) VEGF-B₁₆₇ treatment inhibited the Bmf-induced cellular apoptosis in the RGC5 cells. Scale bar: 100 μm.
(L) VEGF-B₁₆₇ treatment inhibited the expression of both the endogenous (rat) and exogenous (mouse) Bmf expression in the RGC5 cells. * P<0.05, ** P<0.01

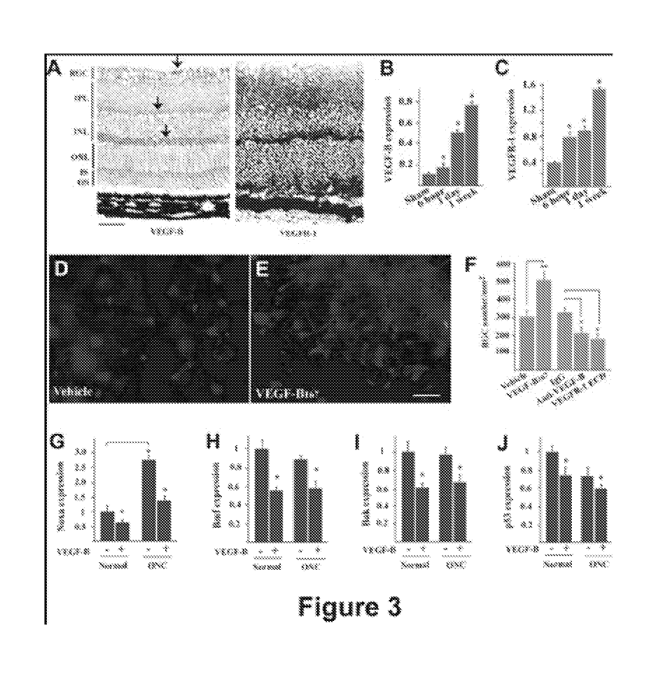

FIG. 3 shows VEGF-B inhibits axotomy-induced apoptosis in the retina.
(A) VEGF-B is highly expressed in the retina as shown by the in situ hybridization assay. High level of VEGF-B expression was found primarily in the retinal ganglion cells (RGC), part of the inner plexiform (IPL) and the inner nuclear layer (INL) (arrows). VEGFR-1 expression was mainly found in the inner plexiform, part of the inner nuclear layer, and the inner and outer segment layers (IS, OS). Scale bar: 50 μm
(B, C) Real-time PCR assay showed that VEGF-B (B) and VEGFR-1 (C) expression were upregulated in the retinae after ONC injury. The upregulation was seen as early as six hours after ONC, and reached a very high level after one week.
(D-F) A single does of VEGF-B₁₆₇ intravitreal treatment increased the number of the viable RGCs by about 1.7 folds. VEGF-B neutralizing antibody intravitreal treatment decreased the number of the viable RGCs by about 33% (F). VEGFR-1 extracellular domain (VEGFR-1 ECD) treatment decreased the number of the viable RGCs by about 42% (F). Scale bar: 10 μm
(G-J) Real-time PCR analysis revealed that VEGF-B₁₆₇ treatment inhibited the expression of the BH3-only protein genes Noxa (G) and Bmf (H), as well as Bak (I) and p53 (J) expression in both normal and ONC-injured retinae. * P<0.05, ** P<0.01

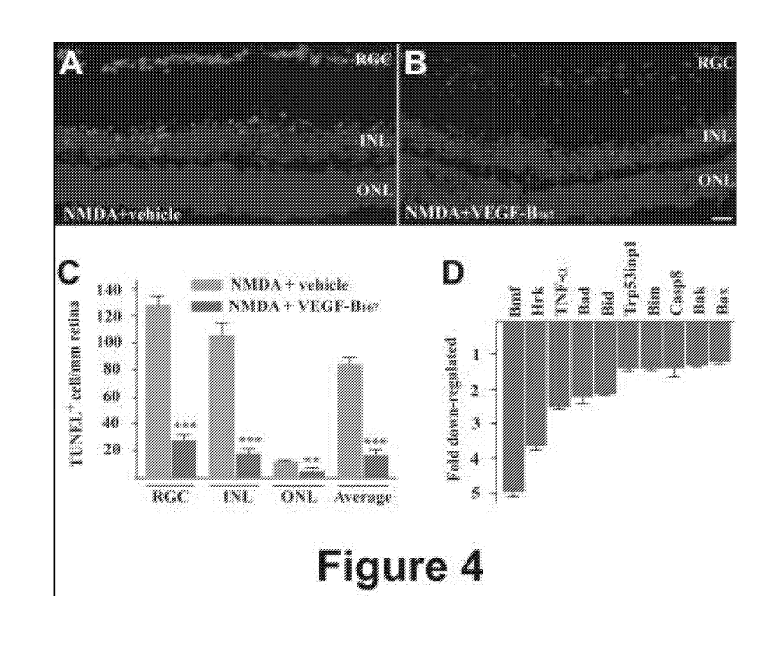

FIG. 4 shows VEGF-B inhibits excitotoxin-induced apoptosis in the retina
(A) N-methyl-D-aspartic acid (NMDA) intravitreous treatment led to massive apoptosis in the retina as shown by the TUNEL staining.
(B, C) VEGF-B₁₆₇ treatment significantly reduced the number of apoptotic cells in all the three layers of the retina. Scale bar: 20 μm
(D) Real-time PCR assay revealed that VEGF-B₁₆₇ treatment inhibited the expression of the BH3-only protein (Bmf, Hrk, Bad, Bid, Bim) and other apoptotic/cell death-related genes in the NMDA-injured retina. ** P<0.01, *** P<0.001

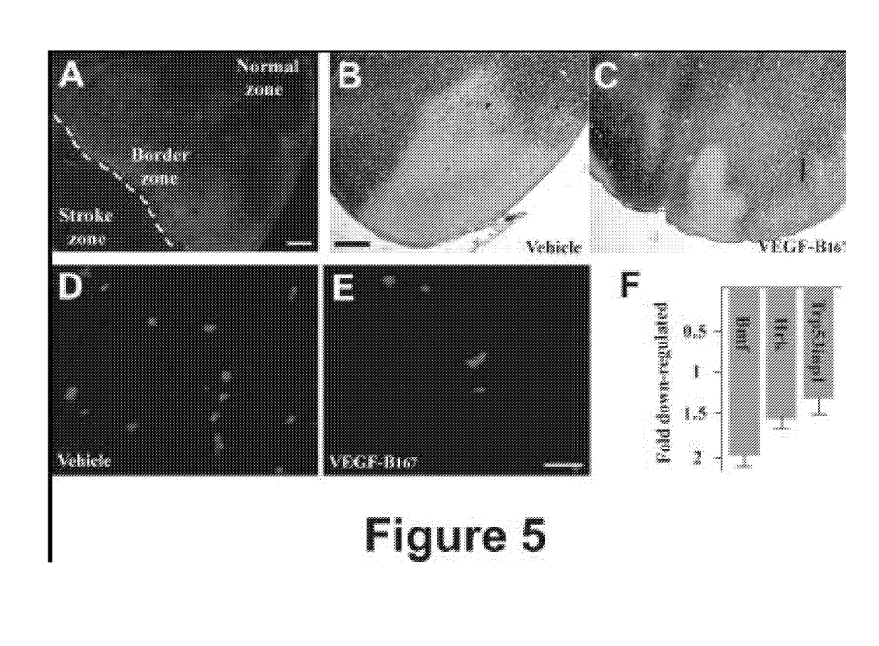

FIG. 5 shows VEGF-B inhibits ischemia-induced neuronal apoptosis and apoptotic gene expression in the brain.
(A) VEGF-B expression is up-regulated in the border zone of the brain after the middle cerebral artery occlusion (MCAO) as shown by the immunohistochemical staining. Scale bar: 50 μm
(B-C) Recombinant human VEGF-B₁₆₇ protein treatment decreased the brain damage volume by about 32% in the wild-type mice as shown by the Map2 staining. Scale bar: 100 μm
(D, E) VEGF-B₁₆₇ treatment decreased the number of the apoptotic cells in the border zone of the stroke as shown by the TUNEL staining. Scale bar: 10 μm
(F) VEGF-B₁₆₇ treatment inhibited the expression of the BH3-only protein genes Bmf, Hrk, and the apoptotic gene Trp53inp1 in the brain with stroke.

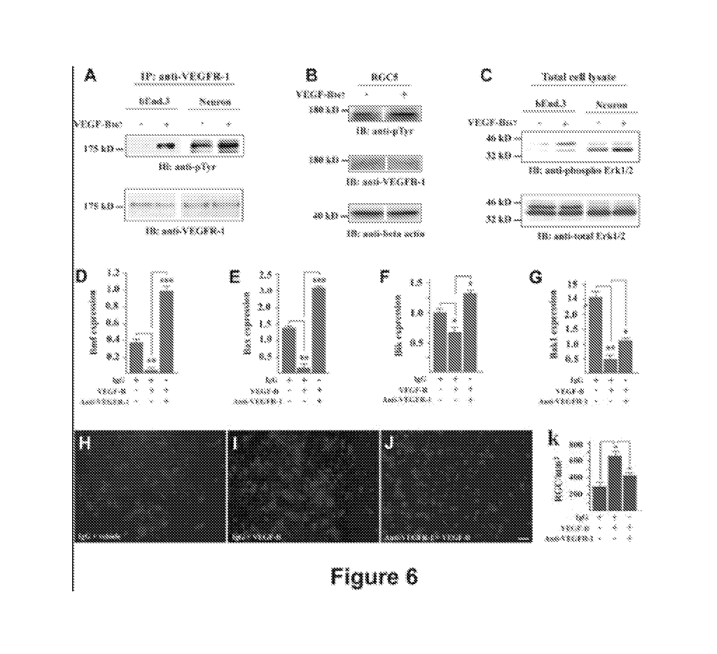

FIG. 6 shows VEGFR-1 mediates the effect of VEGF-B
(A) VEGF-B₁₆₇ stimulation resulted in VEGFR-1 activation in the bEnd.3 cells and the cortex neurons using the immunoprecipitation assay and the anti-VEGFR-1 antibody, followed by the Western blot assay using the anti-phosphotyrosine antibody (anti-pTyr). VEGFR-1 was detected in the bEnd.3 cells and the cortex neurons using the Western blot assay. IP: immunoprecipitation, IB: immunoblotting
(B) VEGF-B₁₆₇ induced VEGFR-1 activation in the RGCS cells using the Western blot assay and the antibodies against the phosphotyrosine and VEGFR-1.
(C) VEGF-B₁₆₇ treatment led to ERK1/2 activation in both the bEnd.3 cells and the cortex neurons using the Western blot assay and antibodies against the phosphorylated and total ERK 1/2.
(D-G) VEGFR-1 neutralizing antibody treatment abolished to various degrees the inhibitory effect of VEGF-B on the expression of Bmf (D), Bax (E), Bak1 (F), and Bik (G) in the RGC5 cells.
(H, I, K) VEGF-B protein treatment increased RGC survival in the ONC-injured retina. Scale bar: 20 μm (J, K) VEGFR-1 neutralizing antibody treatment largely abolished the VEGF-B-induced RGC survival in the ONC-injured retina. * P<0.05, ** P<0.01, *** P<0.001

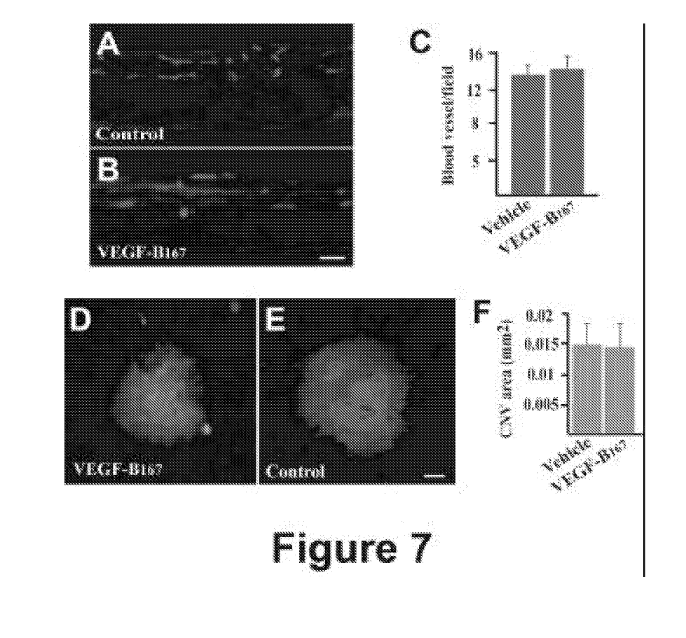

FIG. 7 shows VEGF-B does not affect retinal angiogenesis.
(A-C) VEGF-B₁₆₇ treatment did not affect blood vessel density and morphology in the retina using the isolectin B4 staining. Scale bar: 50 μm (D-F) VEGF-B₁₆₇ intravitreous administration after laser treatment did not change the choroidal neovascularization area as shown by the IB4 staining. Scale bar: 50 μm

### DETAILED DESCRIPTION OF THE INVENTION

The invention generally relates to methods for treating a degenerative disease in a mammalian subject. The method provides administering VEGF-B polypeptide or a functional variant or mimetic thereof in an amount effective to reduce or eliminate the degenerative disease in the mammalian subject. The invention further relates to methods for treating pathological conditions associated with excessive apoptosis in a mammalian subject. The method provides administering VEGF-B polypeptide, or functional variant or mimetic thereof, in an amount effective to reduce or eliminate the diseased conditions in the mammalian subject. The treatment of the degenerative disease or neurodegenerative disease with the VEGF-B polypeptide, or functional variant or mimetic thereof, is effective and provides a superior treatment compared to VEGF, since the treatment which administers a VEGF-B polypeptide or a functional variant or mimetic thereof does not stimulate angiogenesis in the mammalian subject. The disease can be generally related to a condition associated with excessive apoptosis. The disease can include, but is not limited to, neurodegenerative disease or ocular neurodegenerative disease, neurovascular degenerative disease, tissue injury, ischemia, or AIDS. Specific types of disease include, but are not limited to, neurodegenerative disease, neurodegenerative ocular disease, glaucoma, diabetic retinopathy, retinitis pigmentosa, Usher syndrome, cone-rod dystrophies, Stargardt disease, choroideremia, retinoschisis, Leber amaurosis, retinoblastoma, muscular dystrophy, aging, autism, stroke, or brain injury. The disease can be, for example, multiple sclerosis, Alzheimer's disease, amyotrophic lateral sclerosis (ALS), fibromyalgia, tinnitus, Parkinson's disease, or Huntington's disease. The disease can be, for example, a condition associated with excessive apoptosis, AIDS, haematological disorders, aplastic anaemia, G6PD deficiency, myelodysplastic syndrome, T CD4+ lymphocytopenia, pathologies of tissue damage, myocardial infarction, ischemic renal damage, or polycystic kidney disease.

A method for treating a condition associated with apoptosis deficiency in a mammalian subject is provided which comprises administering a VEGF-B antagonist, or functional variant or mimetic thereof, in an amount effective to reduce or eliminate the condition in the mammalian subject. The disease includes, but is not limited to, a neoplastic disease, autoimmune disease, inflammatory disease, or viral infectious disease. The VEGF-B antagonist or functional variant or mimetic can include, but is not limited to, an antibody, RNA, DNA, RNAi, shRNA, antisense nucleic acid, small chemical molecule, or dominant-negative protein. The VEGF-B antagonist functional variant or mimetic can be a conservative amino acid substitution or peptide mimetic substitution. In one aspect, the VEGF-B antagonist can be a neutralizing antibody to VEGF-B. The VEGF-B neutralizing antibody can be a neutralizing antibody as discussed in Example 4. Also see, for example, Scotney et al., Clin. Exp. Pharmacol. Physiol. 29: 1024-1029, 2002.

Despite its early discovery and high sequence homology to the other VEGF family members, the function of VEGF-B has long been a debatable issue. The present invention provides a novel function of VEGF-B as a potent apoptosis inhibitor. VEGF-B treatment rescues neurons from apoptosis in both the retina and brain. A genome-wide gene expression profiling assay was performed to determine the biological function of VEGF-B. The study validated findings in multiple cell lines and animal models. Mechanistically, the present study demonstrated that VEGF-B inhibits the expression of the BH3-only protein and other apoptotic/cell death-related genes via VEGFR-1. Remarkably, the survival effect of VEGF-B is not associated with undesired angiogenesis. VEGF-B appears to be the first member of the VEGF family that has a potent anti-apoptotic effect, while lacking a general angiogenic activity. VEGF-B thus offers a new therapeutic option for the treatment of degenerative diseases, including but not limited to, neurodegenerative diseases.

The present study reports the findings that:
(1) VEGF-B is a potent apoptosis inhibitor, and rescues neurons from apoptosis in both the retina and brain. The study further demonstrated the mechanism underlying the function of VEGF-B by showing that VEGF-B potently inhibits the expression of the BH3-only protein and other apoptotic/cell death-related genes via VEGFR-1. This was observed not only in neurons, but also in many other types of cells, indicating the generality of this mechanism.
(2) The study further showed that the anti-apoptotic effect of VEGF-B is not associated with undesired angiogenesis. This nature of VEGF-B is of particular importance for the potential clinical usage of VEGF-B as a neuroprotective factor. Even though other VEGF family members have been shown to have neural protective effect, they in the meantime also stimulate undesired angiogenesis and blood vessel permeability, which are highly detrimental. VEGF-B, however, has no effect on blood vessel permeability, and did not affect angiogenesis in the models tested. VEGF-B thus offers a new and better therapeutic option for the treatment of neurodegenerative diseases.

This study used comprehensive experimental approaches, including the genome-wide gene expression microarray assay, multiple cultured cell lines, and several animal models using both the VEGF-B deficient and wild-type mice, to delineate the biological function of VEGF-B.

The present study:
(1) revealed a novel function of VEGF-B;
(2) provided new insights into the mechanisms underlying the function of VEGF-B;
(3) demonstrated that VEGF-B may be of significant therapeutic value for the treatment of neurodegenerative diseases.

It is to be understood that this invention is not limited to particular methods, reagents, compounds compositions or biological systems, which can, of course, vary. It is also to be understood that the terminology used herein is for the purpose of describing particular aspects only, and is not intended to be limiting. As used in this specification and the appended claims, the singular forms “a,” “an” and “the” include plural referents unless the content clearly dictates otherwise. Thus, for example, reference to “a cell” includes a combination of two or more cells, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“VEGF-B,” as used herein encompasses those polypeptides identified as VEGF-B in U.S. Pat. No. 6,331,301, which is incorporated herein in its entirety, as well as published US-A-2003/0008824. and International Patent Application WO 96/26736 which corresponds to U.S. Pat. No. 5,928,939 and in U.S. Pat. No. 5,840,693 and U.S. Pat. No. 5,607,918.

VEGF-B includes, but is not limited to, both the VEGF-B₁₆₇ and/or VEGF-B₁₈₆ isoforms or a fragment, analog or derivative thereof having the ability to bind VEGFR-1. Active analogs should exhibit at least 85% sequence identity, preferably at least 90% sequence identity, particularly preferably at least 95% sequence identity, and especially preferably at least 98% sequence identity to the natural VEGF-B polypeptides, as determined by BLAST analysis. The active substance typically will include the amino acid sequence Pro-Xaa-Cys-Val-Xaa-Xaa-Xa-a-Arg-Cys-Xaa-Gly-Cys-Cys (SEQ ID NO:1) (where Xaa may be any amino acid) that is characteristic of VEGF-B.

Use of polypeptides comprising VEGF-B sequences modified with conservative substitutions, insertions, and/or deletions, but which still retain the biological activity of VEGF-B is within the scope of the invention. Standard methods can readily be used to generate such polypeptides including site-directed mutagenesis of VEGF-B polynucleotides, or specific enzymatic cleavage and ligation. Similarly, use of peptidomimetic compounds or compounds in which one or more amino acid residues are replaced by a non-naturally-occurring amino acid or an amino acid analog that retains the required aspects of the biological activity of VEGF-B is contemplated. Active analogs should exhibit at least 85% sequence identity, preferably at least 90% sequence identity, particularly preferable at least 95% sequence identity, and especially preferable at least 98% sequence identity to the natural VEGF-B polypeptides, as determined by BLAST analysis.

In addition, variant forms of VEGF-B polypeptides that may result from alternative splicing and naturally-occurring allelic variation of the nucleic acid sequence encoding VEGF-B are useful in the present methods. Allelic variants are well known in the art, and represent alternative forms or a nucleic acid sequence that comprise substitution, deletion or addition of one or more nucleotides, but which do not result in any substantial functional alteration of the encoded polypeptide.

Variant forms of VEGF-B can be prepared by targeting non-essential regions of a VEGF-B polypeptide for modification. These non-essential regions are expected to fall outside the strongly-conserved regions of the VEGF family of growth factors. In particular, the growth factors of the VEGF family, including VEGF-B are dimeric, and at least VEGF-A, VEGF-B, VEGF-C, and VEGF-D show complete conservation of eight cysteine residues in the N-terminal domains, i.e. the VEGF-like domains. [Olofsson, et al., Proc. Nat'l. Acad. Sci. USA, 93:2576-2581 (1996); Joukov, et al., EMBO J., 15:290-298 (1996).] These cysteines are thought to be involved in intra- and inter-molecular disulfide bonding. In addition there are further strongly, but not completely, conserved cysteine residues in the C-terminal domains. Loops 1, 2 and 3 of each subunit, which are formed by intra-molecular disulfide bonding, are involved in binding to the receptors for the VEGF family of growth factors. [Andersson, et al., Growth Factors, 12:159-64 (1995).]

These conserved cysteine residues are preferably preserved in any proposed variant form, although there may be exceptions, because receptor-binding VEGF-B analogs are known in which one or more of the cysteines is not conserved. Similarly, the active sites present in loops 1, 2 and 3 also should be preserved. Other regions of the molecule can be expected to be of lesser importance for biological function, and therefore offer suitable targets for modification. Modified polypeptides can readily be tested for their ability to show the biological activity of VEGF-B by routine activity assay procedures such as a VEGFR-1 binding assay or a stem cell proliferation assay based on the examples set forth below.

Preferably, where amino acid substitution is used, the substitution is conservative, i.e. an amino acid is replaced by one of similar size and with similar charge properties.

VEGF-B protein can be modified, for instance, by glycosylation, amidation, carboxylation, or phosphorylation, or by the creation of acid addition salts, amides, esters, in particular C-terminal esters, and N-acyl derivatives. The proteins also can be modified to create peptide derivatives by forming covalent or noncovalent complexes with other moieties. Covalently bound complexes can be prepared by linking the chemical moieties to functional groups on the side chains of amino acids comprising the peptides, or at the N- or C-terminus.

VEGF-B proteins can be conjugated to a reporter group, including, but not limited to a radiolabel, a fluorescent label, an enzyme (e.g., that catalyzes a calorimetric or fluorometric reaction), a substrate, a solid matrix, or a carrier (e.g., biotin or avidin).

Examples of VEGF-B analogs are described in WO 98/28621 and in Olofsson, et al., Proc. Nat'l. Acad. Sci. USA, 95:11709-11714 (1998), both incorporated herein by reference.

VEGF-B polypeptides are preferably produced by expression of DNA sequences that encode them such as DNAs that correspond to, or that hybridize under stringent conditions with the compliments of VEGF-B DNA sequences. Suitable hybridization conditions include, for example, 50% formamide, 5×SSPE buffer, 5×Denhardts solution, 0.5% SDS and 100 μg/ml of salmon sperm DNA at 42° C. overnight, followed by washing 2×30 minutes in 2×SSC at 55° C. Such hybridization conditions are applicable to any polynucleotide encoding one or more of the VEGF-B growth factors or analogs or derivatives thereof.

The VEGF-B as described herein is also directed to an isolated and/or purified DNA that corresponds to, or that hybridizes under stringent conditions with, any one of the foregoing DNA sequences.

The VEGF-B proteins and polypeptides for use in the present invention are characterized by the amino acid sequence Pro-Xaa-Cys-Val-Xaa-Xaa-Xaa-Arg-Cys-Xaa-Gly-Cys-Cys (SEQ ID NO:1) and having the property of inhibiting apoptosis in the treatment of a degenerative disease wherein the protein comprises a sequence of amino acids substantially corresponding to an amino acid sequence of VEGF-B or functional variant or mimetic thereof. VEGF-B dimers may comprise VEGF-B polypeptides of identical sequence, of different VEGF-β isoforms, or other heterogeneous VEGF-B molecules.

The VEGF-B for use according to the present invention can be used in the form of a protein dimer comprising VEGF-B protein, particularly a disulfide-linked dimer. The protein dimers of the invention include both homodimers of VEGF-B and heterodimers of VEGF-B and VEGF polypeptides, as well as other VEGF family growth factors including, but not limited to placental growth factor (P1GF), which are capable of binding to VEGFR-1 (fit-1). The VEGF-B as described herein also includes VEGF-B polypeptides that have been engineered to contain a N-glycosylation cite such as those described in Jeltsch, et al., WO 02/07514, which is incorporated herein in its entirety. Preparation of VEGF-B is discussed in U.S. Pat. No. 6,331,301, which is incorporated herein in its entirety.

“Vector” refers to a nucleic acid molecule amplification, replication, and/or expression vehicle in the form of a plasmid or viral DNA system where the plasmid or viral DNA may be functional with bacterial, yeast, invertebrate, and/or mammalian host cells. The vector may remain independent of host cell genomic DNA or may integrate in whole or in part with the genomic DNA. The vector will contain all necessary elements so as to be functional

“Patient,” “vertebrate subject” or “mammalian subject” are used herein and refer to mammals such as human patients and non-human primates, as well as experimental animals such as rabbits, rats, and mice, and other animals. Animals include all vertebrates, e.g., mammals and non-mammals, such as sheep, cows, dogs, cats, avian species, chickens, amphibians, reptiles, osteichthyes, or chondrichthyes.

“Treating” or “treatment” includes the administration of the compositions, compounds or agents of aspects of the present invention to prevent or delay the onset of the symptoms, complications, or biochemical indicia of a disease, alleviating or ameliorating the symptoms or arresting or inhibiting further development of the disease, condition, or disorder (degenerative disease, e.g., a neuro-vascular degenerative disease). “Treating” further refers to any indicia of success in the treatment or amelioration or prevention of the disease, condition, or disorder (degenerative disease, e.g., a neuro-vascular degenerative disease), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of aspects of the present invention to prevent or delay, to alleviate, or to arrest or inhibit development of the symptoms or conditions associated with a degenerative disease, e.g., a neuro-vascular degenerative disease. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of degenerative disease, e.g., a neuro-vascular degenerative disease but does not yet experience or exhibit symptoms, inhibiting the symptoms of a degenerative disease, e.g., a neuro-vascular degenerative disease (slowing or arresting its development), providing relief from the symptoms or side-effects of degenerative disease, e.g., a neuro-vascular degenerative disease (including palliative treatment), and relieving the symptoms of the degenerative disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Degenerative disease” refers to an ischemic disease, neurodegenerative disease, neurodegenerative ocular disease, or neuro-vascular degenerative disease. Degenerative diseases include, but are not limited to, glaucoma, diabetic retinopathy, retinitis pigmentosa, Usher syndrome, cone-rod dystrophies, Stargardt disease, choroideremia, retinoschisis, Leber amaurosis, retinoblastoma, muscular dystrophy, aging, autism, stroke, brain injury, multiple sclerosis, Alzheimer's disease, amyotrophic lateral sclerosis (ALS), fibromyalgia, tinnitus, Parkinson's disease, or Huntington's disease.

“Ischemic disease” refers to a pathological condition caused by hypoxia due to inadequate blood circulation to a tissue due to blockage of the blood vessels to the area. Diseases include, but, are not limited to, ischemic heart disease, ischemic stroke, ischemic cardiomyopathy, ischemic colitis, bowel, ischemic optic neuropathy, myocardial infarction, ischemic renal damage, or polycystic kidney disease.

Hereditary and degenerative diseases of the central nervous system include, but are not limited to, cerebral degenerations usually manifest in childhood, such as leukodystrophy, Krabbe disease, Pelizaeus-Merzbacher disease, cerebral lipidoses, Tay-Sachs disease; Other cerebral degenerations, such as Alzheimer's, Pick's disease, obstructive hydrocephalus, cerebral degeneration, Reye's syndrome; Parkinson's disease, primary parkinsonism. Extrapyramidal disease and abnormal movement disorders system degenerative diseases of the basal ganglia, Olivopontocerebellar atrophy, Shy-Drager syndrome, essential tremor/familial tremor, myoclonus, Lafora's disease, Unverricht disease, Huntington's chorea, fragments of torsion dystonia, blepharospasm, unspecified extrapyramidal diseases and abnormal movement disorders, extrapyramidal diseases and abnormal movement disorders, restless legs, AND orserotonin syndrome.

Hereditary and degenerative diseases of the central nervous system include, but are not limited to, spinocerebellar disease include, but are not limited to, Friedreich's ataxia, spinocerebellar ataxia, hereditary spastic paraplegia, primary cerebellar degeneration, other cerebellar ataxia, cerebellar ataxia, ataxia-telangiectasia [Louis-Bar syndrome], or corticostriatal-spinal degeneration. Anterior horn cell disease include, but are not limited to, motor neuron disease, amyotrophic lateral sclerosis, progressive muscular atrophy, progressive bulbar palsy, pseudobulbar palsy, primary lateral sclerosis, and other motor neuron diseases. Other diseases of spinal cord include syringomyelia and syringobulbia. Disorders of the autonomic nervous system, include those of the cardiovascular system (such as neurally mediated syncope, autonomic neuropathy, vagally mediated atrial fibrillation, catecholamine-sensitive tachycardias, sudden cardiac death, long QT syndrome, and inappropriate sinus tachycardia), autonomic neuropathy disorders, sympathetic nervous system disorders, parasympathetic nervous system disorders, dysautonomia, Horner's syndrome, neuropathy, vascular neuropathy, gastroparesis, diabetic gastroparesis, diabetic diarrhea, and bladder neuropathy.

A “naturally-occurring” nucleic acid molecule refers to an RNA or DNA molecule having a nucleotide sequence that occurs in nature (e.g., encodes a natural protein).

A “naturally-occurring” polypeptide or protein refers to a polypeptide molecule having an amino acid sequence that occurs in nature (e.g., encodes a natural protein).

“Gene” and “recombinant gene” refer to nucleic acid molecules which include an open reading frame encoding a VEGF-B polypeptide, or mimetic, analog or derivative thereof, preferably a vertebrate, mammalian, bovine, human, avian reptilian, amphibian, osteichthyes, or chondrichthyes peptide, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one aspect, the language “substantially free” means a preparation of a VEGF-B polypeptide, or mimetic, analog or derivative thereof, having less than about 30%, 20%, 10% and more preferably 5% (by dry weight), of non-VEGF-B protein (also referred to herein as a “contaminating protein”). When the VEGF-B polypeptide, or mimetic, analog or derivative thereof, or biologically active portion thereof, is recombinantly produced, it is also preferably substantially free of culture medium, i.e., culture medium represents less than about 20%, more preferably less than about 10%, and most preferably less than about 5% of the volume of the protein preparation. Aspects of the invention include isolated or purified preparations of at least 0.01, 0.1, 1.0, and 10 milligrams in dry weight.

A “non-essential” amino acid residue is a residue that can be altered from the wild-type sequence of the VEGF-B polypeptide, or mimetic, analog or derivative thereof, without abolishing or more preferably, without substantially altering a biological activity, whereas an “essential” amino acid residue results in such a change. For example, amino acid residues that are conserved among the VEGF-B polypeptides, or mimetic, analog or derivative thereof, those present in the domain of VEGF-B polypeptide necessary for anti-apoptotic activity, are predicted to be particularly not amenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a VEGF-B polypeptide, or mimetic, analog or derivative thereof, is preferably replaced with another amino acid residue from the same side chain family. Alternatively, in another aspect, mutations can be introduced randomly along all or part of a VEGF-B polypeptide coding sequence, such as by saturation mutagenesis, and the resultant mutants can be screened for VEGF-B biological activity to identify mutants that retain activity. Following mutagenesis of a VEGF-B polypeptide, or mimetic, analog or derivative thereof, the encoded polypeptide can be expressed recombinantly and the activity of the protein can be determined.

“Biologically active,” when used in conjunction with VEGF-B refers to a VEGF-B polypeptide that binds VEGFR-1 (also known as fit-1) in a manner substantially similar to that of full length VEGF-B, and/or that inhibits apoptosis or apoptotic gene expression.

A biologically active portion of VEGF-B polypeptide can be a polypeptide which is, for example, 10, 25, 50, 100, 200, or more, amino acids in length. Biologically active portions of a VEGF-B polypeptide can be used as targets for developing agents which modulate a VEGF-B activity as described herein.

Calculations of homology or sequence identity (the terms are used interchangeably herein) between sequences are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred aspect, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the VEGF-B polypeptide amino acid sequence, or mimetic, analog or derivative thereof, at least 10, preferably at least 20, more preferably at least 50, even more preferably at least 100 amino acid residues of the two sequences are aligned. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred aspect, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred aspect, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of aspects of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM 120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules encoding a modified VEGF-B polypeptide, or mimetic, analog or derivative thereof, of aspects of the invention. BLAST protein searches can be performed with the XBLAST program, score=50, wordlength=3 to obtain amino acid sequences homologous to VEGF-B polypeptide of aspects of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particular VEGF-B polypeptide, or mimetic, analog or derivative thereof, in aspects of the present invention have an amino acid sequence sufficiently identical or substantially identical to the amino acid sequence of the VEGF-B polypeptide. “Sufficiently identical” or “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

A “purified preparation of cells,” as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An intact “antibody” comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind VEGF Receptor-1 (VEGFR-1) polypeptide or VEGF-B polypeptide. Examples of binding include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., Nature 341: 544-546, 1989), which consists of a VH domain; and (vi) an isolated complementarity determining region (CDR).

An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

“Fab antibodies” or “Fab fragments” refers to antibody fragments lacking all or part of an immunoglobulin constant region, and containing the Fab regions of the antibodies. Fab antibodies are prepared as described herein.

“Single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., Science 242: 423-426, 1988; and Huston et al., Proc. Natl. Acad. Sci. USA, 85: 5879-5883, 1988). Such single chain antibodies are included by reference to the term “antibody” fragments can be prepared by recombinant techniques or enzymatic or chemical cleavage of intact antibodies.

“Human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT Publication Nos. WO 01/14424 and WO 00/37504. However, the term “human sequence antibody,” as used herein, is not intended to include antibodies in which CDR sequences derived from the germline of another mammalian species, such as a mouse, have been grafted onto human framework sequences (e.g., humanized antibodies).

Also, recombinant immunoglobulins may be produced. See, Cabilly, U.S. Pat. No. 4,816,567, incorporated herein by reference in its entirety and for all purposes; and Queen et al., Proc. Nat'l Acad. Sci. USA 86: 10029-10033, 1989.

“Monoclonal antibody” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Polyclonal antibody” refers to a preparation of more than 1 (two or more) different antibodies to a cell surface receptor or a ligand, e.g., VEGF Receptor-1 (VEGFR-1) polypeptide or VEGF-B polypeptide. Such a preparation includes antibodies binding to a range of different epitopes. Antibodies to VEGF Receptor-1 (VEGFR-1) polypeptide or VEGF-B polypeptide can bind to an epitope on VEGF Receptor-1 polypeptide or polypeptide so as to activate or mimic VEGF-B binding to VEGFR-1. These and other antibodies suitable for use in the present invention can be prepared according to methods that are well known in the art and/or are described in the references cited here. In preferred embodiments, anti-VEGF Receptor-1 (VEGFR-1) polypeptide or anti-VEGF-B polypeptide antibodies used in the invention are “human antibodies,” e.g., antibodies isolated from a human—or they are “human sequence antibodies”.

### Methods of Treatment

Candidate conditions for anti-apoptotic treatment with VEGF-B thus include, inter alia, methods for preventing or treating a degenerative disease, e.g., a neuro-vascular degenerative disease in a mammalian subject. VEGF-B polypeptide, or mimetic, analog or derivative thereof, can be administered intraocularly to treat optic neuropathies or intrathecally into neurons, spinal cord, or the brain. VEGF-B can be administered by direct muscle or myocardial injection of naked plasmid DNA encoding VEGF-B during surgery in patients with muscular dystrophy or myocardial ischemia following procedures outlined in Vale, P. R., et al., Circulation, 2000 102 965-74.

VEGF-B can also be administered by direct neural or muscular injection of VEGF-B polypeptide, or mimetic, analog or derivative thereof. Preferably, it is given as a bolus dose of from 1 μg/kg to 15 mg/kg, preferably between 5 μg/kg and 5 mg/kg, and most preferably between 0.2 and 2 mg/kg. Continuous infusion may also be used, for example, by means of an osmotic minipump as described in Heyman et al., Nat Med, 1999 5 1135-152. If so, the medicament may be infused at a dose between 5 and 20 μg/kg/minute, preferably between 7 and 15 μg/kg/minute.

Another possibility for VEGF-B administration is injection of VEGF-B plasmid into apoptotic nervous tissue intraocularly or intrathecally to spinal cord or brain, or in the muscles of an ischemic limb in accordance with procedures described in Simovic, D., et al., Arch Neurol, 2001 58(5) 761-68.

Still another technique for effective VEGF-B administration is by intraocular, intrathecal, or intraarterial gene transfer of the VEGF-B gene using adenovirus and replication defective retroviruses as described in Baumgartner I and Isner J M, Annu Rev Physiol, 2001 63 427-50.

An additional possibility for administering VEGF-B is by intraocular, intrathecal, intramuscular or intravenous administration of recombinant VEGF-B protein. A still further possibility is to use ex vivo expanded neural progenitor cells (NPCs) or endothelial progenitor cells (EPCs) engineered to express VEGF-B in neuronal cells or muscle cells. Kawamoto, A., et al., Circulation, 2001 103(5) 634-37.

Yet another technique which may be used to administer VEGF-B is percutaneous adenovirus-mediated VEGF-B gene delivery to neuronal cells or muscle cells. See, for example, Maillard, L., et al., Gene Ther, 2000 7(16) 1353-61; and Laham R J, Simons M, and Sellke F, Annu Rev Med, 2001 52 485-502.

In one advantageous aspect, a therapeutically effective dose of VEGF-B is administered by bolus injection of the active substance into apoptotic or ischemic tissue, e.g., neural tissue, or heart or peripheral muscle tissue. The effective dose will vary depending on the weight and condition of the ischemic subject and the nature of the ischemic condition to be treated. It is considered to be within the skill of the art to determine the appropriate dosage for a given subject and condition.

In accordance with another aspect, VEGF-B is administered by continuous delivery, e.g., using an osmotic minipump, until the patient is able to self-maintain a functional neuronal tissue or muscle.

In another advantageous aspect, VEGF-B is effectively administered to a subject having optical neuropathy, brain or spinal neuropathy, or ischemic tissue by contacting the tissue with a viral vector, e.g., an adenovirus vector, containing a polynucleotide sequence encoding VEGF-B operatively linked to a promoter sequence.

VEGF-B may also be effectively administered by implantation of a micropellet impregnated with active substance in the direct vicinity of optical neuropathy, brain or spinal neuropathy, or ischemic tissue.

### Peptides and Polypeptides

Aspects of the invention provide isolated or recombinant polypeptides comprising an amino acid sequence having at least 95%, 96%, 97%, 98%, 99% or more sequence identity to VEGF-B polypeptide over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of aspects of the invention. Aspects of the invention provide methods for preventing or treating a degenerative disease, e.g., a neuro-vascular degenerative disease in a mammalian subject comprising administering to the vertebrate subject a VEGF-B polypeptide, or mimetic, analog or derivative thereof. A VEGF-B polypeptide, or mimetic, analog or derivative thereof, in an aspect of the invention that inhibits apoptotic activity, or inhibits apoptotic gene expression can reduce or eliminate degenerative disease, e.g., a neuro-vascular degenerative disease.

In one aspect, an aspects of the invention provides VEGF-B polypeptide, or mimetic, analog or derivative thereof, and the nucleic acids encoding them where one, some or all of the VEGF-B polypeptide, is replaced with substituted amino acids. In one aspect, an aspects of the invention provides methods to inhibit apoptotic activity, or inhibits apoptotic gene expression to reduce or eliminate degenerative disease, e.g., a neuro-vascular degenerative disease.

The VEGF-B polypeptide, or mimetic, analog or derivative thereof, in aspects of the invention can be expressed recombinantly in vivo after administration of nucleic acids, as described above, or, they can be administered directly, e.g., as a pharmaceutical composition.

VEGF-B polypeptide, or mimetic, analog or derivative thereof, in aspects of the invention can be isolated from natural sources, be synthetic, or be recombinantly generated polypeptides. Peptides and proteins can be recombinantly expressed in vitro or in vivo. The VEGF-B polypeptide in aspects of the invention can be made and isolated using any method known in the art. VEGF-B polypeptide in aspects of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, A. K., Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The VEGF-B polypeptide, or mimetic, analog or derivative thereof, in aspects of the invention, as defined above, includes all “mimetic” and “peptidomimetic” forms. The terms “mimetic” and “peptidomimetic” refer to a synthetic chemical compound which has substantially the same structural and/or functional characteristics of the polypeptides of aspects of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the structure and/or activity of the mimetic. As with polypeptides of aspects of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has a VEGF-B polypeptide activity.

In one aspect, the polypeptide or peptidomimetic composition can be a dominant-negative mutant within the scope of the invention if it can inhibit apoptotic activity. The dominant negative mutant can be a peptide or peptide mimetic that can inhibit degenerative disease, e.g., a neuro-vascular degenerative disease, or a nucleic acid composition, in the form of a DNA vector or gene therapy vector, that expresses a dominant-negative polypeptide that can inhibit degenerative disease, e.g., a neuro-vascular degenerative disease. The dominant negative mutant can bind to a ligand of the VEGF-B receptor, NP-1. The dominant negative molecule can act, for example, by interfering with protein interactions.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy guanidino, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of guanidino and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a VEGF-B polypeptide, or mimetic, analog or derivative thereof, in aspects of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form.

Various chemical modifications will improve the stability, bioactivity and ability of the VEGF-B polypeptide, or mimetic, analog or derivative thereof, to cross the blood brain barrier to treat degenerative disease, e.g., a neuro-vascular degenerative disease. One such modification is aliphatic amino terminal modification with a derivative of an aliphatic or aromatic acid, forming an amide bond. Such derivatives include, for example, CH₃CO, CH₃(CH₂)ₙCO, C₆H₅CH₂CO and H₂N(CH₂)ₙCO, wherein n=1-10. Another modification is carboxy terminal modification with a derivative of an aliphatic or aromatic amine/alcohol coupled to the VEGF-B polypeptide via an amide/ester bond. Such derivatives include those listed above. The VEGF-B polypeptide may also have both amino and carboxy terminal modifications, wherein the derivatives are independently selected from those listed above. The peptides may also be glycosylated, wherein either the alpha amino group or a D-Asn, or both, are modified with glucose or galactose. In another contemplated modification, selected backbone amide bonds are reduced (—NH—CH₂). Other modifications include N-methylation of selected nitrogens in the amide bonds and esters in which at least one of the acid groups on the peptide are modified as aromatic or aliphatic esters. Any combination of the above modifications is also contemplated.

Aspects of the invention also provide polypeptides that are “substantially identical” to an exemplary polypeptide of aspects of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a VEGF-B polypeptide, or mimetic, analog or derivative thereof, of aspects of the invention, resulting in modification of the structure of the polypeptide, without significantly altering its biological activity. For example, amino- or carboxyl-terminal, or internal, amino acids which are not required for a VEGF-B activity or interaction can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of aspects of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of aspects of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

A VEGF-B polypeptide, or mimetic, analog or derivative thereof, in aspects of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Inc., Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

“Polypeptide” and “protein” as used herein, refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The VEGF-B polypeptide, or mimetic, analog or derivative thereof, in aspects of the invention also include all “mimetic” and “peptidomimetic” forms, as described in further detail, below.

“Isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, aspects of the invention provide nucleic acids which have been purified from genomic DNA or from other sequences in a library or other environment by at least one, two, three, four, five or more orders of magnitude.

### Therapeutic Applications

The VEGF-B polypeptide, or mimetic, analog or derivative thereof, identified by the methods as described herein can be used in a variety of methods of treatment of degenerative disease, e.g., a neuro-vascular degenerative disease. Thus, the present invention provides compositions and methods for treating a neuro-vascular degenerative disease. The composition includes a VEGF-B polypeptide, or mimetic, analog or derivative thereof, and a pharmaceutically acceptable carrier. The VEGF-B composition can be administered alone or in combination with other compositions.

A VEGF-B polypeptide, or mimetic, analog or derivative thereof, as described herein, can be used in methods for preventing or treating a degenerative disease, e.g., a neuro-vascular degenerative disease in a mammalian subject. VEGF-B does not have a general effect on angiogenesis and blood vessel permeability. This nature of VEGF-B is of particular importance for the potential clinical usage of VEGF-B as a neuroprotective factor. VEGF-B thus has an unusual safety profile with minimum side effect as a survival molecule. It may therefore be used to treat a broad array of neurodegenerative diseases involving neuronal death, such as ocular neurodegenerative diseases, glaucoma stroke, autism, multiple sclerosis, Alzheimer's disease, amyotrophic lateral sclerosis (ALS), fibromyalgia, tinnitus, Parkinson's disease, and Huntington's disease. VEGF-B therefore offers a new and better therapeutic option for the treatment of neurodegenerative diseases.

Preferably, treatment using VEGF-B polypeptide, or mimetic, analog or derivative thereof, in an aspect of the present invention could either be by administering an effective amount of the VEGF-B polypeptide to the patient. Moreover, the polypeptide or peptidomimetic as provided herein can be used to reduce or eliminate degenerative disease, e.g., a neuro-vascular degenerative disease.

Hereditary and degenerative diseases of the central nervous Cerebral degenerations usually manifest in childhood, such as leukodystrophy, Krabbe disease, Pelizaeus-Merzbacher disease, cerebral lipidoses, Tay-Sachs disease; Other cerebral degenerations, such as Alzheimer's, Pick's disease, obstructive hydrocephalus, cerebral degeneration, Reye's syndrome; Parkinson's disease, primary parkinsonism. Extrapyramidal disease and abnormal movement disorders system degenerative diseases of the basal ganglia, Olivopontocerebellar atrophy, Shy-Drager syndrome, essential tremor/familial tremor, myoclonus, Lafora's disease, Unverricht disease, Huntington's chorea, fragments of torsion dystonia, blepharospasm, unspecified extrapyramidal diseases and abnormal movement disorders, extrapyramidal diseases and abnormal movement disorders, restless legs, orserotonin syndrome

Spinocerebellar disease include, but are not limited to, Friedreich's ataxia, spinocerebellar ataxia, hereditary spastic paraplegia, primary cerebellar degeneration, other cerebellar ataxia, cerebellar ataxia, ataxia-telangiectasia [Louis-Bar syndrome], or corticostriatal-spinal degeneration. Anterior horn cell disease include, but are not limited to, Motor neuron disease, Amyotrophic lateral sclerosis, Progressive muscular atrophy, Progressive bulbar palsy, Pseudobulbar palsy, Primary lateral sclerosis, Other motor neuron diseases. Other diseases of spinal cord include Syringomyelia and syringobulbia. Disorders of the autonomic nervous system include Reflex sympathetic dystrophy.

### Pharmaceutical Compositions

A VEGF-B polypeptide, or mimetic, analog or derivative thereof, useful in the present compositions and methods can be administered to a human patient per se, in the form of a stereoisomer, prodrug, pharmaceutically acceptable salt, hydrate, solvate, acid salt hydrate, N-oxide or isomorphic crystalline form thereof, or in the form of a pharmaceutical composition where the compound is mixed with suitable carriers or excipient(s) in a therapeutically effective amount, for example, to treat degenerative disease, e.g., a neuro-vascular degenerative disease.

“Therapeutically effective amount” refers to that amount of the therapeutic agent, VEGF-B polypeptide, or mimetic, analog or derivative thereof, sufficient to result in the amelioration of one or more symptoms of a disorder, or prevent advancement of a disorder, cause regression of the disorder, or to enhance or improve the therapeutic effect(s) of another therapeutic agent. With respect to the treatment of degenerative disease, e.g., a neuro-vascular degenerative disease, a therapeutically effective amount refers to the amount of a therapeutic agent sufficient to reduce or eliminate neurodegenerative disease. Preferably, a therapeutically effective amount of a therapeutic agent reduces or eliminates a degenerative disease, e.g., a neuro-vascular degenerative disease, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. With respect to the treatment of a degenerative disease, e.g., a neuro-vascular degenerative disease, a therapeutically effective amount refers to the amount of a therapeutic agent that reduces the disease by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. “Therapeutic protocol” refers to a regimen for dosing and timing the administration of one or more therapeutic agents, such as a VEGF-B polypeptide, or mimetic, analog or derivative thereof.

Pharmaceutically acceptable carriers are determined in part by the particular composition being administered, as well as by the particular method used to administer the composition. Accordingly, there is a wide variety of suitable formulations of pharmaceutical compositions for administering the antibody compositions (see, e.g., latest edition of Remington's Pharmaceutical Sciences, Mack Publishing Co., Easton, Pa., incorporated herein by reference). The pharmaceutical compositions generally comprise a differentially expressed protein, VEGF-B polypeptide, or mimetic, analog or derivative thereof, in a form suitable for administration to a patient. The pharmaceutical compositions are generally formulated as sterile, substantially isotonic and in full compliance with all Good Manufacturing Practice (GMP) regulations of the U.S. Food and Drug Administration.

### Treatment Regimes

Aspects of the invention provide pharmaceutical compositions comprising one or a combination of VEGF-B polypeptide, or mimetic, analog or derivative thereof, formulated together with a pharmaceutically acceptable carrier. Some compositions include a combination of multiple (e.g., two or more) VEGF-B polypeptide, or mimetic, analog or derivative thereof.

In prophylactic applications, pharmaceutical compositions or medicaments are administered to a patient susceptible to, or otherwise at risk of a disease or condition (i.e., degenerative disease, e.g., a neuro-vascular degenerative disease) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, the immune response is monitored and repeated dosages are given if the immune response starts to wane.

### Effective Dosages

Effective doses of the VEGF-B polypeptide, or mimetic, analog or derivative thereof, for the treatment of degenerative disease, e.g., a neuro-vascular degenerative disease, as described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with a VEGF-B polypeptide, or mimetic, analog or derivative thereof, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more VEGF-B polypeptides, or mimetic, analog or derivative thereof, with different binding specificities are administered simultaneously, in which case the dosage of each VEGF-B polypeptideis usually administered on multiple occasions. Intervals between single dosages can be weekly, monthly or yearly. Intervals can also be irregular as indicated by measuring blood levels of VEGF-B polypeptide in the patient. In some methods, dosage is adjusted to achieve an concentration of 1-1000 μg/ml VEGF-B polypeptide and in some methods 25-300 μg/ml. Alternatively, VEGF-B polypeptide, or mimetic, analog or derivative thereof can be administered as a sustained release formulation, in which case less frequent administration is required. Dosage and frequency vary depending on the half-life of the compound in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of degenerative disease, e.g., a neuro-vascular degenerative disease. Thereafter, the patent can be administered a prophylactic regime.

Doses for a nucleic acid vector encoding VEGF-B polypeptide, or mimetic, analog or derivative thereof, range from about 10 ng to 1 g, 100 ng to 100 mg, 1 μg to 10 mg, or 30-300 μg DNA per patient. Doses for infectious viral vectors vary from 10-100, or more, virions per dose.

### Prodrugs

The present invention is also related to prodrugs of the agents obtained by the methods disclosed herein. Prodrugs are agents which are converted in vivo to active forms (see, e.g., R. B. Silverman, 1992, The Organic Chemistry of Drug Design and Drug Action, Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids.

Examples of prodrugs and their uses are well known in the art (see, e.g., Berge et al., “Pharmaceutical Salts,” J. Pharm. Sci. 66: 1-19, 1977). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

### Routes of Administration

A VEGF-B polypeptide, or mimetic, analog or derivative thereof, for treatment or amelioration of degenerative disease, e.g., a neuro-vascular degenerative disease can be administered by parenteral, topical, intravenous, oral, subcutaneous, intraarterial, intracranial, intraperitoneal, intranasal or intramuscular means for prophylactic as inhalants for VEGF-B polypeptide, or mimetic, analog or derivative thereof, preparations targeting degenerative disease, e.g., a neuro-vascular degenerative disease in neuronal tissue, or muscle tissue, and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where a tumor is found, for example intracranial injection or convection enhanced delivery. Intramuscular injection or intravenous infusion are preferred for administration of antibody. In some methods, particular therapeutic antibodies are delivered directly into the cranium. In some methods, antibodies are administered as a sustained release composition or device, such as a Medipad™ device.

Agents of the invention can optionally be administered in combination with other agents that are at least partly effective in treating degenerative disease, e.g., a neuro-vascular degenerative disease. In the case of disease in the brain, agents of the invention can also be administered in conjunction with other agents that increase passage of the agents of the invention across the blood-brain barrier (BBB) to treat neuro-vascular degenerative disease.

### Formulation

A VEGF-B polypeptide, or mimetic, analog or derivative thereof, for the treatment of degenerative disease, e.g., a neuro-vascular degenerative disease, are often administered as pharmaceutical compositions comprising an active therapeutic agent, i.e., and a variety of other pharmaceutically acceptable components. See latest edition of Remington's Pharmaceutical Science (Mack Publishing Company, Easton, Pa.). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of aspects of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### Toxicity

Preferably, a therapeutically effective dose of VEGF-B polypeptide, or mimetic, analog or derivative thereof, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1,

### Kits

Also within the scope of the invention are kits comprising a VEGF-B polypeptide, or mimetic, analog or derivative thereof, of aspects of the invention and instructions for use. The kit can further contain a least one additional reagent, or one or more additional human antibodies of aspects of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

The invention will be further described with reference to the following examples; however, it is to be understood that the invention is not limited to such examples.

The following examples of specific aspects for carrying out the present invention are offered for illustrative purposes only, and are not intended to limit the scope of the present invention in any way.

### EXAMPLES

### Example 1

### Genome-Wide Gene Profiling Reveals that VEGF-B Inhibits the Expression of Numerous Apoptotic/Cell Death-Related Genes

To determine the biological function of VEGF-B, the genome-wide gene expression profiling assay was performed to identify the genes regulated by VEGF-B. The mouse primary aortic smooth muscles cells was treated with human recombinant VEGF-B₁₆₇ protein in a hypoxic condition (1% oxygen), because these cells express VEGFR-1, and VEGFR-1 is upregulated in hypoxia. The expression profiling analysis was performed using the whole mouse genome microarray containing 41,534 mouse genes and transcripts. Results revealed that VEGF-B treatment significantly down-regulated the expression of about six hundred genes by more than two-fold. Among them, the most significantly down-regulated genes fall into the apoptosis/cell death-related pathways (Table 1). The top-three most down-regulated genes were the ones critically involved in apoptosis: Bmf (29) (about ten-fold down-regulated), TrP53inp1 (30) (about eight-fold down-regulated), and DCN (31) (about seven-fold down-regulated). Thus, the genes that are most significantly down-regulated by VEGF-B are the ones critically involved in the apoptosis/cell death-related pathways.

TABLE 1
Apoptotic/cell death-related genes down-regulated by VEGF-B₁₆₇

| Gene Symbol | Accession Number | Fold down-regulated |
|---|---|---|
| Bmf | AK040622 | 9.60 |
| Trp53inp1 | NM_021897 | 7.90 |
| Dcn | NM_007833 | 7.34 |
| Angpt2 | NM_007426 | 4.98 |
| Sesn1 | BC055753 | 4.80 |
| Txnip | AK004653 | 4.62 |
| Cables1 | NM_022021 | 4.55 |
| Aox1 | NM_009676 | 3.98 |
| Abtb1 | NM_030251 | 3.69 |
| Ptpn13 | NM_011204 | 3.35 |
| Map3k7ip2 | NM_138667 | 3.10 |
| Noxa | NM_021451 | 2.95 |
| Scn2a1 | AK089150 | 2.80 |
| Mbd4 | NM_010774 | 2.75 |
| Ikbkg | AK042138 | 2.55 |
| Casp2 | NM_007610 | 2.42 |
| Prkdc | NM_011159 | 2.40 |
| Aim1 | NM_172393 | 2.26 |
| Rbl2 | NM_011250 | 2.30 |
| Map3k1 | NM_011945 | 2.23 |
| Aatk | NM_007377 | 2.20 |
| Plagl1 | BC065150 | 2.19 |
| Tlr2 | NM_011905 | 2.19 |
| Adrb2 | NM_007420 | 2.16 |
| Cradd | NM_009950 | 2.12 |
| Ar | NM_013476 | 2.11 |
| Dedd2 | AK010701 | 2.10 |
| Itgb3bp | AK088352 | 2.10 |
| Thap3 | NM_175152 | 2.10 |
| Ifih1 | NM_027835 | 2.00 |

### Example 2

### VEGF-B Inhibits Apoptotic Gene Expression in Multiple Cell Lines

Four different cell lines were next used to verify the microarray data. The rat retinal ganglion cell-derived cell line RGC5, the immortalized rat retinal pericyte cell line TR-rPCT, the immortalized rat retinal Müller cell line TR-MUL, and the immortalized rat retinal endothelial cell line TR-iBRB were treated with VEGF-B₁₆₇ (100 ng/ml) for six hours in either normoxic or hypoxic (1% oxygen) conditions, and the expression of the apoptotic/cell death-related genes investigated by real-time PCR assay. VEGFR-1 expression was detected by real-time PCR assay in all the four cell lines. VEGF-B significantly inhibited the expression of many apoptotic/cell death-related genes, consistent with the microarray data. Among these genes, the expression of most of the BH3-only protein genes (Bmf, Hrk, Puma, Noxa (Pmaip1), Bad, Bid, Bik) was inhibited by VEGF-B in all of the four different cell lines (FIG. 1A-D). The BH3-only protein genes are therefore the major target genes suppressed by VEGF-B. VEGF-B also inhibited the expression of p53, the caspases (Casp2, Casp8, Casp9, Casp12), and other apoptotic/cell death-related genes (Bak, Bax, Bcl2l11, TNF-α, Dcn, Olr1, FIG. 1A-D). Thus, VEGF-B inhibited the expression of the BH3-only protein and other apoptotic/cell death-related genes in all of the four types of cell lines investigated, indicating a general role of VEGF-B as an apoptosis inhibitor in different types of cells.

### Example 3

### VEGF-B Inhibits Oxidative Stress-, Serum Deprivation-, and Bmf-Induced Apoptosis in Cultured Cells

Different approaches were next used to test whether VEGF-B could inhibit apoptosis in the RGC5 cells. We first treated the RGC5 cells with hydrogen peroxide (H₂O₂), which is known to induce oxidative stress-induced apoptosis, and investigated the apoptosis status using the terminal deoxynucleotidyl transferase dUTP nick end labeling (TUNEL) assay. VEGF-B₁₆₇ treatment (100 ng/ml) significantly decreased the H₂O₂-induced apoptosis in the RGC5 cells by about 50% (FIG. 2A-D, n=3, P<0.01). We next tested whether VEGF-B could inhibit the serum deprivation-induced cell death in the RGC5 cells. The cells were cultured in serum-free medium and treated the cells with recombinant proteins of human VEGF-B₁₆₇ (100 ng/ml), VEGF (25 and 100 ng/ml) and P1GF (25 and 100 ng/ml), and the viability of the cells measured at different time point using the MTT assay. VEGF-B treatment significantly increased cell survival as potently as 10% FCS at day one and two (FIG. 2E, n=3, P<0.01). The survival effect of VEGF-B₁₆₇ on the RGC5 cells was slightly greater in a hypoxic condition. In contrast, P1GF, another VEGFR-1 ligand, did not show such an effect (FIG. 2F). VEGF had only a weak survival effect on the RGC5 cells (FIG. 2G, n=3, P<0.05).

Bmf is an essential apoptosis inducer in response to cellular stresses (29). Over-expression of Bmf led to cellular apoptosis within twenty-four hours (29). The potent inhibitory effect of VEGF-B on the expression of Bmf indicates that VEGF-B may inhibit Bmf-mediated apoptosis. To test this, the mouse Bmf gene was overexpressed in the rat RGC5 cells, and determined the apoptosis rate and the expression of Bmf level in the cells with or without VEGF-B treatment. Bmf overexpression induced significantly cellular apoptosis in the RGC5 cells (FIG. 2H, I, K, n=3, P<0.01). VEGF-B167 treatment (100 ng/ml for forty-eight hours) inhibited the Bmf-induced apoptosis by about 40% (FIG. 2J, K, n=3, P<0.01). Further, VEGF-B treatment inhibited the expression of both the endogenous rat and the exogenous mouse Bmf genes (FIG. 2L, n=3, P<0.01). Thus, VEGF-B inhibited the oxidative stress-, serum deprivation-, and Bmf-induced apoptosis in the RGC5 cells.

### Example 4

### VEGF-B Inhibits Axotomy-Induced Neuronal Apoptosis and Apoptotic Gene Expression in the Retina

The anti-apoptotic effect of VEGF-B in vivo was investigated using several different animal models. First, we used the optic nerve crush (ONC) injury mouse model. In this model, it is known that ONC injury results in apoptotic death of the retinal ganglion cells (RGCs) in the retina after the crush injury. In situ hybridization analysis showed that both VEGF-B and VEGFR-1 are highly expressed in the retina (FIG. 3A). Higher level of VEGF-B expression was found in the retinal ganglion cells (RGC), part of the inner plexiform, and cells in the inner nuclear layer (FIG. 3A, right, arrows). VEGFR-1 expression was mainly found in the inner plexiform, part of the inner nuclear layer, and the rod and cone layer (FIG. 3A, left). Real-time PCR assay showed that both VEGF-B (FIG. 3B) and VEGFR-1 (FIG. 3C) were significantly upregulated in the retinae after ONC injury. The upregulation was seen as early as six hours after ONC, and reached a very high level at one week after ONC injury (FIG. 3B, C, n=4, P<0.05). To test whether VEGF-B can rescue RGC death, human recombinant VEGF-B₁₆₇ was administered intravitreally into the mouse eyes after ONC injury, and counted the viable RGCs after two weeks. Treatment with a single dose of VEGF-B₁₆₇ (500 ng/eye) significantly increased the number of the viable RGCs by about 1.7-fold (FIG. 3D-F, n=8, 12, P<0.01). On the contrary, intravitreal treatment with VEGF-B neutralizing antibody (32) (5 pmol/eye) decreased the number of the viable RGCs by about 33% (FIG. 3F, n=8, P<0.05). Since it is known that VEGF-B binds to VEGFR-1 (11), we next tested whether inhibition of VEGF-B by the VEGFR-1 extracellular domain (VEGFR-1 ECD, 5 pmol/eye), acting as a ligand trap, could impair RGC survival. VEGFR-1 ECD treatment decreased the viable RGCs by about 42% (FIG. 3F, n=8, P<0.05). Thus, VEGF-B is critically required for RGC survival in the injured retina.

To clarify the molecular mechanism underlying the survival effect of VEGF-B in the retina, the expression of an array of apoptotic/cell death-related genes in the retinae was investigated with or without ONC injury and with or without VEGF-B treatment one week after the injury. Real-time PCR analysis revealed that VEGF-B treatment (500 ng/eye) inhibited the expression of the BH3-only protein genes Noxa, Bmf, as well as the apoptotic genes Bak and p53 in both normal and ONC-injured retinae (FIG. 3G-J, n=4, P<0.05). It is interesting to note that Noxa expression in the retina was upregulated to about 2.7-fold one week after the ONC injury, while no significant difference was observed with the other apoptotic genes (FIG. 3G-J). VEGF-B thus promotes RGC survival, at least partially, by inhibiting the expression of the BH3-only protein and other apoptotic genes in the retina.

### Example 5

### VEGF-B Inhibits N-Methyl-D-Aspartic Acid (NMDA)-Induced Neuronal Apoptosis and Apoptotic/Cell Death-Related Gene Expression in the Retina

In many neurodegenerative and neurologic disorders, neuronal injury and death are caused by the overstimulation of the excitatory amino acids receptors. We next tested whether VEGF-B could inhibit the excitotoxin-induced apoptosis in the retina using the NMDA injury model. NMDA (20 nmol/eye) was injected with or without VEGF-B₁₆₇ (500 ng/eye) intravitreally into the mouse eyes. After twenty-four hours, the retinae were harvested for TUNEL staining and gene expression analysis. VEGF-B₁₆₇ treatment significantly reduced the number of apoptotic cells in the RGC, the inner nuclear, and the outer nuclear layers (FIG. 4A-C, n=8, P<0.001 and 0.01). Real-time PCR assay revealed that VEGF-B treatment inhibited the expression of a number of the BH3-only protein genes (Bmf, Hrk, Bad Bid, Bim), as well as other apoptotic/cell death-related genes (TNF-α, Trp53inp1, Casp8, Bak, Bax) in the NMDA-injured retina (FIG. 4D). VEGF-B thus is a potent inhibitor of the excitotoxin-induced apoptosis, at least partially, via suppression of the BH3-only protein and other apoptotic/cell death-related gene expression.

### Example 6

### VEGF-B Inhibits Ischemia-Induced Neuronal Apoptosis and Apoptotic Gene Expression in the Brain

It has been shown that VEGF-B deficiency led to enlarged brain infarct volume after middle cerebral artery occlusion in mouse (33). However, the molecular mechanisms underlying remain unclear. Further, it remains unknown whether VEGF-B protein treatment could rescue the ischemia-induced neuronal death in the brain. The middle cerebral artery occlusion (MCAO) stroke model was performed using both the VEGF-B deficient and wild-type mice to address these questions. Immunohistochemical staining showed that both VEGF-B and VEGFR-1 are expressed in the neurons in normal brain. The VEGF-B expression was highly up-regulated in the border zone of the brain after stroke (FIG. 5A). VEGF-B deficiency led to about 50% larger brain damage volume as compared with the wild-type mice (Table 2, n=10, P<0.05). This defect was largely rescued by VEGF-B protein treatment (Table 2, n=9, 10, P<0.05). It is interesting to note that in the wild-type mice, even though the endogenous VEGF-B is highly expressed in the brain (FIG. 5A), VEGF-B protein treatment still decreased the stroke volume by about 32% (Table 2, n=8, 10, P<0.05; FIG. 5B-C). TUNEL staining showed that VEGF-B₁₆₇ treatment significantly decreased the number of the apoptotic cells in the border zone of the stroke, where the surviving neurons were endangered to apoptosis (number of apoptotic cells/field: 31±9 in the control group versus 12±1 in the VEGF-B treatment group, n=3, P<0.05; FIG. 5D, E). VEGF-B treatment thus protected neurons from apoptosis in the brain. To understand the molecular mechanisms underlying, we analyzed the expression of the BH3-only protein and other apoptotic genes in the brains with MCAO with or without VEGF-B treatment. VEGF-B treatment significantly down-regulated the expression of the BH3-only protein genes Bmf, Hrk, and the apoptotic gene Trp53inp1 using the real-time PCR assay (FIG. 5F). Thus, VEGF-B protects the neurons in the brain, at least partially, by inhibiting the expression of the BH3-only protein and other apoptotic/cell death-related genes.

TABLE 2
VEGF-B treatment reduces brain damage volume
in both VEGF-B deficient and wild-type mice

| | Stroke volume (mm³) |
|---|---|
| VEGF-B KO | 18.9 ± 1.9 |
| WT | 12.8 ± 1.8 |
| VEGF-B KO + VEGF-B | 10.7 ± 2 |
| VEGF-B KO + vehicle | 15.9 ± 1.7 |
| WT + VEGF-B | 7.7 ± 1.3 |
| WT + vehicle | 11.3 ± 1 |

### Example 7

### VEGFR-1 Mediates the Anti-Apoptotic Effect of VEGF-B

It is known that VEGF-B binds to VEGFR-1 (11). To verify whether the effect of VEGF-B is mediated by VEGFR-1, it was determined whether VEGFR-1 could be activated by VEGF-B in different types of cells. Using Western blot assay, VEGFR-1 was detected in the cortex neurons isolated from neonatal mice, in the bEnd.3 cell line (transformed mouse cerebral cortex-derived endothelial cells), and in the RGC5 cells (FIG. 6A, B) Immunoprecipitation using an anti-VEGFR-1 antibody followed by Western blot assay using an anti-phosphotyrosine antibody showed that VEGF-B₁₆₇ stimulation (100 ng/ml) resulted in VEGFR-1 activation in the bEnd.3 cells, cortex neurons, and the RGC5 cells (FIG. 6A, B). The cell lysate was further subjected to Western blot assay using antibodies against the phosphorylated and total ERK1/2. In both the bEnd.3 cells and the cortex neurons, VEGF-B treatment led to ERK1/2 activation (FIG. 6C). Thus, VEGF-B₁₆₇ treatment activated VEGFR-1 followed by the activation of the ERK1/2 pathway in different types of cells.

We next tested whether VEGFR-1 blockade could abolish the inhibitory effect of VEGF-B on the expression of the apoptotic/cell death-related genes in the RGC5 cells. VEGFR-1 neutralizing antibody treatment abolished completely the inhibitory effect of VEGF-B on the expression of Bmf, Bax, and Bik, indicating that the effect of VEGF-B indeed was mediated by VEGFR-1 (FIG. 6D-F, n=3, P<0.001 and 0.05 respectively). Moreover, VEGFR-1 neutralizing antibody treatment up-regulated the expression of Bmf, Bax, and Bik (FIG. 6D-F), indicating that other VEGFR-1 ligands than VEGF-B may also contribute to the inhibition of their expression via VEGFR-1. In addition, VEGFR-1 neutralizing antibody treatment abolished the inhibitory effect of VEGF-B on the expression of Bak1 to a lesser extent (FIG. 6G, n=3, P<0.05). Thus, the inhibitory effect of VEGF-B on the expression of the BH3-only protein and other apoptotic/cell death-related genes is largely mediated by VEGFR-1. Indeed, this observation was confirmed in vivo using the ONC retinal injury model. When the VEGF-B protein was administered together with the VEGFR-1 neutralizing antibody into the mouse vitreous with ONC injury, the VEGF-B-induced RGC survival was largely abolished (FIG. 6H-K, n=8, P<0.05). Thus, in vivo data confirmed that the anti-apoptotic effect of VEGF-B is mediated by the VEGFR-1.

### Example 8

### VEGF-B does not Induce Angiogenesis and Blood Vessel Permeability

Over-growth of blood vessels in the eye may lead to severe vision damage or loss. It was therefore investigated whether VEGF-B₁₆₇ intravitreal administration could affect normal retinal vasculature and pathological angiogenesis. The retinae were sectioned with or without VEGF-B₁₆₇ treatment and stained them with isolectin B4 (IB4), which labels the vascular endothelial cells, and blood vessel density was counted in different locations of the retina (central, middle and peripheral). No difference was observed in terms of blood vessel density and morphology fourteen days after VEGF-B₁₆₇ treatment (FIG. 7A-C, n=6, P>0.05). Thus, VEGF-B protein treatment does not affect normal blood vessels in the retina. To investigate whether VEGF-B affects pathological angiogenesis, we performed the laser-induced choroidal neovascularization (CNV) model. VEGF-B₁₆₇ was administered intravitreally immediately after laser treatment. One week after VEGF-B₁₆₇ treatment, when choroidal neovascularization reaches the peak level, the eyes were harvested and the CNV area measured after IB4 staining. No difference was observed between the VEGF-B₁₆₇-treated and the control groups (FIG. 7D-F, n=8, P>0.05). To test whether VEGF-B is involved in blood vessel permeability, we performed the modified Mile's assay using the VEGF-B deficient and wild-type mice. No difference was found between the two groups (OD620: 0.17±0.01 in VEGF-B deficient and 0.19±0.02 in wild-type mice, n=10, 12, P>0.05). Thus, VEGF-B did not affect normal or pathological angiogenesis and blood vessel permeability in the model systems tested.

### Example 9

### VEGF-B is an Apoptosis Inhibitor by Suppression of BH3-Only Protein Gene Expression Via VEGFR-1

VEGF-B was discovered many years ago, and has a high sequence homology to the other VEGF family members. However, the function of VEGF-B is still a debatable issue. We reveal here for the first time a novel function of VEGF-B as a potent apoptosis inhibitor. VEGF-B treatment rescues neurons from apoptosis in both the retina and brain. Mechanistically, we demonstrate that VEGF-B inhibits the expression of the BH3-only protein and other apoptotic/cell death-related genes via VEGFR-1. Most interestingly, the survival effect of VEGF-B is not associated with undesired angiogenesis. VEGF-B thus appears to be the first member of the VEGF family that has a potent anti-apoptotic effect, while lacking a general angiogenic activity.

Even though VEGF has also been shown to be a potent neuroprotective factor (34), the therapeutic potential of its neuroprotective effect is limited because of its potent and general angiogenic and permeability-promoting effect. In contrast to the other VEGF family members, VEGF-B does not have a general effect on angiogenesis and blood vessel permeability. This nature of VEGF-B is of particular importance for the potential clinical usage of VEGF-B as a neuroprotective factor. VEGF-B thus has an unusual safety profile with minimum side effect as a survival molecule. It may therefore be used to treat a broad array of neurodegenerative diseases involving neuronal death, such as ocular neurodegenerative diseases, stroke, autism, multiple sclerosis, Alzheimer's disease, amyotrophic lateral sclerosis (ALS), fibromyalgia, tinnitus, Parkinson's disease, and Huntington's disease. VEGF-B therefore offers a new and better therapeutic option for the treatment of neurodegenerative diseases.

Most of the VEGF family members have been shown to play critical roles in development or physiological/pathological angiogenesis (4, 15, 35, 36). It is therefore surprising that VEGF-B did not seem to have a major impact during development, physiological or pathological angiogenesis (8, 9), even though it has a high sequence similarity to the other VEGF family members and uses the same receptors. Indeed, in contrast to VEGF and VEGF-C deficient mice (15, 35, 36), which are embryonic lethal, the VEGF-B-deficient mice are largely normal, regarding their lifespan, fertility, angiogenesis, (16, 17). In contrast to P1GF, which is critically required for pathological angiogenesis (4), VEGF-B is not required for neovessel formation in different pathological conditions (16, 18-21, 27). In contrast to the other VEGF family members, which all stimulate blood vessel leakage (37-39), VEGF-B has no effect on blood vessel permeability (19, 22-24). In contrast to VEGF, which is a potent stimulator for vascular endothelial cell (EC) proliferation and migration, VEGF-B has no effect on EC proliferation and migration (9). Several studies have reported an angiogenic activity of VEGF-B (24-26). However, this has not been observed by other investigators (16, 18, 20, 21, 27). Thus, studies on VEGF-B have led to debatable and inconsistent results, and the function of VEGF-B remains elusive.

This study demonstrated that VEGF-B signaling is critical for cell survival. Using the genome-wide gene profiling approach, we revealed that the genes most significantly down-regulated by VEGF-B are the genes that are critically involved in the apoptosis/cell death-related pathways. We validated our results both in vitro and in vivo using multiple cell lines and animal models. Among the genes down-regulated by VEGF-B, the BH3-only proteins, which are critical regulators in initiating apoptosis, were consistently inhibited by VEGF-B in different experimental settings. The BH3-only proteins are therefore the main targets suppressed by VEGF-B, suggesting a major role of VEGF-B in inhibiting apoptosis. Indeed, using different animal models including the optic nerve crush injury- and the NMDA-induced retinal neuron apoptosis model, as well as the acute ischemia-induced brain neuron apoptosis model, we confirmed that VEGF-B inhibits apoptosis and promotes neuronal survival in both the retina and brain, in both the axotomy-, neurotoxin-, and the ischemia-induced neuronal death, at least partially, by inhibiting the expression of the BH3-only protein and other apoptotic/cell death-related genes. The BH3-only proteins are the key upstream regulators in initiating apoptosis. Their activities are mainly regulated at the transcription level. By inhibiting the expression of the BH3-only protein family members and other apoptotic/cell death-related genes, VEGF-B keeps apoptosis in check at an early stage.

VEGF-B binds to VEGFR-1 and neuropilin-1 (NP-1) (10, 11). VEGFR-1 is highly expressed in the retina, and is critically required for the maintenance and survival of the retinal vasculature (40). VEGFR-1 is expressed in the brain and up-regulated after brain ischemia (41), suggesting a role of VEGFR-1 in neuronal survival. Indeed, the survival effect of VEGFR-1 has been shown in numerous cell types (42-44). In this study, VEGFR-1 ECD treatment decreased the viable RGCs by about 42% in the ONC injury model, suggesting the critical role of the VEGFR-1 ligands in RGC survival. Indeed, VEGF-B stimulation led to VEGFR-1 and ERK MAP kinase activation in the RGCS, the bEnd.3, and the cortex neuronal cells. Furthermore, VEGFR-1 neutralizing antibody largely abolished the inhibitory effect of VEGF-B on the expression of the apoptotic/cell death-related genes in the RGCS cells in vitro, and decreased the VEGF-B-dependant RGC survival in the retina in vivo. Thus, the anti-apoptotic/survival effect of VEGF-B is mainly mediated by VEGFR-1. The other receptor used by VEGF-B, NP-1, is also expressed by neurons in the retina and brain (45, 46), and is up-regulated after focal cerebral ischemia (46). NP-1 has been shown to promote the survival of various types of cells (47). NP-1 could therefore also be involved in mediating the survival effect of VEGF-B. Further studies are needed to verify this possibility.

In summary, using multiple approaches involving the genome-wide gene profiling assay, cultured cells and animal models, we show for the first time that VEGF-B is a potent apoptosis inhibitor. VEGF-B rescues neurons from apoptosis in both the retina and brain without causing undesired angiogenesis. Moreover, the survival effect of VEGF-B is achieved by inhibiting the expression of the BH3-only protein and other apoptotic/cell death-related genes via VEGFR-1. VEGF-B appears to be the first member of the VEGF family that has a potent anti-apoptotic effect but lacking a general angiogenic activity. VEGF-B thus provides a new therapeutic option for treating different types of neural degenerative diseases.

### Example 10

### Methods and Materials

Microarray analysis. Mouse aortic artery smooth muscle cells (SMCs) were isolated and cultured as described previously (48). SMCs were grown to 80% confluence and synchronized by overnight serum starvation followed by stimulation with human VEGF-B₁₆₇ (100 ng/ml) for six hours in a hypoxic (1% oxygen) condition. Total RNA was isolated using the TRIzol reagent (Invitrogen) followed by the RNeasy Mini kit (Qiagen) purification according to the manufacturer's instruction. Total RNA (10 μg) was subjected to cDNA preparations using the Cy3-dUTP or Cy5-dUTP (Amersham) and an oligo-dT primer. Microarray assay was performed using the Whole Mouse Genome Oligo Microarray (Agilent Technologies). This array is composed of 41,534 (60-mer) oligonucleotide probes representing over 41,000 mouse genes and transcripts compiled from a broad sequence collection. For the first slide, the VEGF-B167 treated cDNA was labeled with the Cy3 fluorescent dye, and the control cDNA was labeled with the Cy5 fluorescent dye. For the second slide, the VEGF-B₁₆₇ treated cDNA was labeled with the Cy5 fluorescent dye, and the control cDNA was labeled with the Cy3 fluorescent dye. The whole experiment, including the treatment of the SMCs with VEGF-B₁₆₇, was repeated independently twice. Thus, four Agilent slides were used and four sets of original signals were obtained with each RNA sample. Two tailed Student's t-test was used for the statistical analysis of the expression data. The threshold of differential gene expression was set at greater than two-fold up-regulation or 50% down-regulation of the genes. Functional grouping of the differentially expressed genes was performed using several different tools including the WebGestalt (http://bioinfo.vanderbilt.edu/webgestalt) and the Ingenuity Pathways Analysis (https://analysis.ingenuity.com/pa/login/login.jsp).

Cell culture, cell viability, Bmf overexpression and Real-time PCR assay. The rat retinal ganglion cell derived cell line RGCS cells, the immortalized rat retinal pericyte cell line (TR-rPCT), and the immortalized rat retinal Müller cell line (TR-MUL) were cultured in DMEM containing 10% fetal bovine serum (FBS) and 1% penicillin/streptomycin at 33° C. with 5% CO₂. The immortalized rat retinal capillary endothelial cell line (TR-iBRB) and the bEnd.3 cell line derived from the transformed mouse cerebral cortex endothelial cells were cultured in DMEM supplemented with endothelial cell growth factors (Roche, 1-033484). For the viability assay, RGCS cells were starved in serum-free medium for 3 days. The cells were then seeded in 96-well tissue culture plate (BD) and allowed to attach for 24 hours in serum-free medium. The medium was then replaced with 100 μl of serum-free medium supplemented with recombinant human VEGF-B₁₆₇ protein (100 ng/ml). Serum-free medium with 100 ng/ml BSA was used as a negative control. Cell culture medium with 10% FBS was used as a positive control. All the experiments were performed in triplicates. Cell viability was evaluated using the MTT method according to the manufacturer's protocol (MTT Cell Viability Assay Kit, Invitrogen) at different time points. After lysis of the cells, the formazan dye was quantified by measuring the absorbance at 540 nm using a photometer (Multiscan EX Microplate Photometer, Thermo). The mouse Bmf expression construct was a kind gift from Dr. Andreas Strasser at The Walter and Eliza Hall Institute of Medical Research, Australia. The DNA plasmid was transfected into the RGCS cells using the FuGENE Transfection Reagent (Roche) according to the manufacturer's instruction. Apoptosis of the transfected cells was analyzed two days after transfection. For VEGFR-1 neutralizing antibody (R&D) treatment, the RGCS cells were treated with the antibody at 500 ng/ml for overnight, followed by VEGF-B recombinant protein treatment at 100 ng/ml for six hours. For the real-time PCR assay, total RNA was isolated using the RNeasy Mini-kit (Qiagen) according to the manufacturer's instruction. 3 μg of total RNA was used for cDNA synthesis using the SuperMix kit (Invitrogen) and used for the Real-time PCR reaction using an ABI Prism 7500 HT Sequence Detection System (Applied Biosystems). All experiments were performed in triplicates and repeated at least twice. Primers used are listed in the supplementary Table 1.

Neonatal neuron isolation and survival assay. For neonatal neuron isolation, cerebral cortex from 1 day-old neonatal mice were collected in Neurobasal A media (Invitrogen) with 0.1% EDTA and digested with trypsin (Invitrogen) for 30 minutes at 37° C., followed by density gradient centrifugation with 7.0, 9.4, 11.9, 16.4% of Optiprep (AXIS-SHIELD). Neurons positioned between the second and third layers were collected and cultured in Neurobasal-A for subsequent analysis. Experiments were performed within ten days of culture. For neuronal survival assay, neurons were washed two times with pre-warmed DMEM without glucose (Invitrogen), and then cultured in DMEM with or without 45 mM glucose. Complete Neurobasal-A medium was used as a control for 100% survival rate. For hypoxia, neurons were cultured in the Aeropack Kenki system (Mitsubishi Gas) at 37° C. After 6 hours, media were harvested, 500 μl of 2% Triton X-100 added and kept for 15 minutes at room temperature. Lactate dehydrogenase (LDH) activity was measured in the media and cell lysate. The ratio of the LDH activity from the cell lysate (viable cells) versus the sum of the total LDH activity (from the lysate and the medium) was used as a survival index. Experiments were performed in triplicates and were repeated at least twice.

Optic nerve crush (ONC) injury mouse model. All the animal experiments were approved by the Animal Care and Use Committee (ACUC) at the NEI/NIH, and were performed according to the NIH guidelines and regulations on animal studies. Female mice (8 weeks old) were deeply anesthetized by intraperitoneal injection of xylazine (14 mg/kg) and ketamine (60 mg/kg), and placed in a small stereotactic instrument. The skull was exposed and kept dry and clean using 3% hydrogen peroxide. The bregma was identified and marked. A hole was drilled above the superior colliculus of each hemisphere. Using a stereotactic measuring device and a Hamilton injector, the mice were injected with FluoroGold in the superior colliculus of each hemisphere at a depth of 1.6 mm from the bony surface of the brain, and the incision sutured. Three days after the tracer dye application, the right optic nerve of each mouse was subjected to a crush injury to cause primary damage to the axons. Using a binocular-operating microscope, the conjunctiva of the right eye was incised. The orbital muscles was teased and deflected aside to expose the optic nerve at its exit from the globe. With the aid of the cross-action forceps, the optic nerve was subjected to a severe crush injury 1-2 mm from the eyeball. For VEGF-B treatment, under a dissecting microscope, using a 33-g needle, 1-2 μl of VEGF-B solution (500 ng) with or without VEGFR-1 neutralizing antibody (20 pmol/eye, R&D) was injected into the vitreous chamber in one eye. After two weeks, mice were deeply anesthetized and eyes collected for analysis. Under a fluorescent microscope, eight pictures from the central and peripheral retina respectively were taken, resulting in sixteen pictures per retina using the AxioVision software (Zeiss), and the viable RGCs counted. The average density of the viable RGCs was calculated from the sixteen pictures.

NMDA retina injury model. 8 weeks old female mice were anesthetized as described above. NMDA (20 nmol in 0.5 μl, together with 500 ng VEGF-B₁₆₇ protein or vehicle in 1 μl) was injected into the eye intravitreally. Intravitreal injection was performed using a 33-gauge needle from 0.5 mm behind the limbus in the temporal region of the globe at a 55° angle to the equator. Mice with vitreous hemorrhage, retinal detachment, or lens trauma caused by the injection were excluded from the assay. Retinae were harvested 24 hour after the injection and embedded in OCT and sectioned through out the whole eye for TUNEL staining. For quantitative analysis, eight microscopic fields were photographed using the AxioVision software (Zeiss) from eight sections of the most severely injured parts of the retina, and TUNEL positive nuclei counted and mean value calculated.

Cerebral ischemic stroke model. Focal cerebral ischemia was produced by permanent occlusion of the middle cerebral artery (MCA) as described previously (49). VEGF-B deficient mice were described previously (16). Littermates from the mice that were backcrossed onto the C57B16 background for more than six generations were used for the experiments. Briefly, 8 weeks old male mice were anesthetized and a U-shape incision was made between the left ear and the left eye. A small opening (1-2 mm in diameter) was made in the region over the MCA with a handheld drill. The MCA was ligated and transected distally to the ligation point. After 24 hours, the mice were killed with an overdose of Nembutal (500 mg/kg, Abbott Laboratories) and perfused with 1% PFA. The brains were removed, postfixed in 1% PFA overnight, and subjected to Map-2 immunohistological staining. The infarct volume was defined as the sum of the MAP-2 negative areas of the sections multiplied by their thickness using the AxioVision software (Zeiss). For VEGF-B protein treatment, a skin incision was made at the top of the head of the anesthetized mouse. Four small openings (1 mm in diameter) were made in the skull at −1, 0, 1, 2 mm from the bregma to the lambda and 1 mm left from the bregma. A fine needle (33G) was inserted to the depth of 1.2 mm from the skull surface and 1 μl of 0.55 μg/μl recombinant human VEGF-B₁₆₇ was injected over 5 minutes into each opening and the skin sutured. MCA ligation was then performed as described above.

Laser-induced CNV model and Miles' assay. Ten weeks old female mice were anesthetized and pupils dilated. Mice were positioned on a rack connected to a slit lamp delivery system. Four photocoagulation spots were made (75 μm spot size, 75 ms, 90 mW power, Oculight Infrared Laser System 810 nm, IRIDEX Corporation) in the area surrounding the optic nerve in each eye. The sites were visualized through a handheld contact lens and a viscous surface lubricant. Only laser-induced burns with a bubble formation were included in the study. The mice were given lubricant ophthalmic ointment after laser treatment. Seven days after laser treatment, the eyes were removed, fixed, retina dissected, and choroids isolated and stained with isolectin B4 conjugated with Alexa Fluor® 568 (Invitrogen). The eyecups were flat-mounted in Aquamount with the sclera facing down, and the total neovascular area measured using the AxioVision software (Zeiss) and the mean value per burn presented for each eye. Miles' assay was performed as described previously (24).

TUNEL staining and in situ hybridization. TUNEL assay was performed according to the manufacturer's protocol (Roche). In situ hybridization was performed as described (50) using a mouse VEGF-B antisense riboprobe on 12 μm frozen tissue sections. A sense riboprobe was used as a negative control. The riboprobes were prepared using the T7 and T3 RNA polymerases and the digocigenin-11-UTP (Roche) according to the manufacturer's instruction.

Cloning and expression of human VEGFR-1 extracellular domain (ECD). Human VEGFR-1 (NM_{—}002019) cDNA plasmid was obtained from Dr. Nader Rahimi (Boston University School of Medicine). The 2212 bp (125-861 aa) VEGFR-1 extracellular domain was PCR amplified using the follow primers: forward 5′-CACGGATCCCACAGGATCTAGTTCAGGTTC-3′ (SEQ ID NO:2) (including a BamHI site), reverse 5′-GCGAATTCTTAGTGATGGTGATGGTGATGCAGATTAGACTTGTCCGAGGT-3′ (SEQ ID NO:3) (including an EcoRI site and a C-terminal 6×his tag). The PCR product was cloned into the pAcGP67A Baculovirus transfer vector (BD) and sequencing verified. The expression vector was co-transfected with the linearized baculovirus DNA into Sf9 insect cells and the virus amplified according to the manufacturer's protocol (Pharmingen) and as described previously (51). The recombinant VEGFR-1 ECD was purified using the Ni-NTA agarose according to the manufacturer's protocol (Qiagen).

VEGFR-1 and ERK1/2 expression and activation assay. A rabbit polyclonal antibody raised against the amino acids 23-247 within the extracellular domain of human VEGFR-1 (sc-9029, Santa Cruz) was used to immunoprecipitate VEGFR-1 in the cell lysate. The immunoprecipitate was analyzed by Western blotting using an anti-phosphotyrosine antibody (sc-7020, Santa Cruz) and a rabbit VEGFR-1 affinity-purified polyclonal antibody (sc-316, Santa Cruz) raised against the peptide mapping at the carboxy terminus of human VEGFR-1. Antibodies against the phosphorylated and total ERK1/2 (Cell Signaling) were used in the Western blot assay to detect the ERK1/2 activation.

Statistics. Two tailed Student's t-test was used for statistical analysis. Difference was considered statistically significant when P<0.05. The data are represented as mean±SEM of the number of the determinations. Assays using cultured cells were performed in triplicates.

### REFERENCES

1. Folkman, J. 2007. Angiogenesis: an organizing principle for drug discovery? Nat Rev Drug Discov 6:273-286.
2. Ferrara, N., and Kerbel, R. S. 2005. Angiogenesis as a therapeutic target. Nature 438:967-974.
3. Carmeliet, P., and Jain, R. K. 2000. Angiogenesis in cancer and other diseases. Nature 407:249-257.
4. Luttun, A., Tjwa, M., Moons, L., Wu, Y., Angelillo-Scherrer, A., Liao, F., Nagy, J. A., Hooper, A., Priller, J., De Klerck, B., et al. 2002. Revascularization of ischemic tissues by P1GF treatment, and inhibition of tumor angiogenesis, arthritis and atherosclerosis by anti-Flt1. Nat Med 1:1.
5. Alitalo, K., Tammela, T., and Petrova, T. V. 2005. Lymphangiogenesis in development and human disease. Nature 438:946-953.
6. Carmeliet, P., and Tessier-Lavigne, M. 2005. Common mechanisms of nerve and blood vessel wiring. Nature 436:193-200.
7. Greenberg, D. A., and Jin, K. 2005. From angiogenesis to neuropathology. Nature 438:954-959.
8. Li, X., and Eriksson, U. 2001. Novel VEGF family members: VEGF-B, VEGF-C and VEGF-D. Int J Biochem Cell Biol 33:421-426.
9. Nash, A. D., Baca, M., Wright, C., and Scotney, P. D. 2006. The biology of vascular endothelial growth factor-B (VEGF-B). Pulm Pharmacol Ther 19:61-69.
10. Olofsson, B., Pajusola, K., Kaipainen, A., von Euler, G., Joukov, V., Saksela, O., Orpana, A., Pettersson, R. F., Alitalo, K., and Eriksson, U. 1996. Vascular endothelial growth factor B, a novel growth factor for endothelial cells. Proc Natl Acad Sci USA 93:2576-2581.
11. Olofsson, B., Korpelainen, E., Pepper, M. S., Mandriota, S. J., Aase, K., Kumar, V., Gunji, Y., Jeltsch, M. M., Shibuya, M., Alitalo, K., et al. 1998. Vascular endothelial growth factor B (VEGF-B) binds to VEGF receptor-1 and regulates plasminogen activator activity in endothelial cells. Proc Natl Acad Sci USA 95:11709-11714.
12. Li, X., Aase, K., Li, H., von Euler, G., and Eriksson, U. 2001. Isoform-specific expression of VEGF-B in normal tissues and tumors. Growth Factors 19:49-59.
13. Aase, K., Lymboussaki, A., Kaipainen, A., Olofsson, B., Alitalo, K., and Eriksson, U. 1999. Localization of VEGF-B in the mouse embryo suggests a paracrine role of the growth factor in the developing vasculature. Developmental Dynamics 215:12-25.
14. Ferrara, N., Carver-Moore, K., Chen, H., Dowd, M., Lu, L., O'shea, K. S., Powell-Braxton, L., Hillan, K. J., and Moore, M. W. 1996. Heterozygous embryonic lethality induced by targeted inactivation of the VEGF gene. Nature 380:439-442.
15. Carmeliet, P., Ferreira, V., Breier, G., Pollefeyt, S., Kieckens, L., Gertsenstein, M., Fahrig, M., Vandenhoeck, A., Harpal, K., Eberhardt, C., et al. 1996. Abnormal blood vessel development and lethality in embryos lacking a single VEGF allele. Nature 380:435-439.
16. Aase, K., von Euler, G., Li, X., Ponten, A., Thoren, P., Cao, R., Cao, Y., Olofsson, B., Gebre-Medhin, S., Pekny, M., et al. 2001. Vascular Endothelial Growth Factor-B-Deficient Mice Display an Atrial Conduction Defect. Circulation 104:358-364.
17. Bellomo, D., Headrick, J. P., Silins, G. U., Paterson, C. A., Thomas, P. S., Gartside, M., Mould, A., Cahill, M. M., Tonks, I. D., Grimmond, S. M., et al. 2000. Mice lacking the vascular endothelial growth factor-B gene (Vegfb) have smaller hearts, dysfunctional coronary vasculature, and impaired recovery from cardiac ischemia. Circulation Research 86:E29-E35.
18. Reichelt, M., Shi, S., Hayes, M., Kay, G., Batch, J., Gole, G. A., and Browning, J. 2003. Vascular endothelial growth factor-B and retinal vascular development in the mouse. Clin Experiment Ophthalmol 31:61-65.
19. Louzier, V., Raffestin, B., Leroux, A., Branellec, D., Caillaud, J. M., Levame, M., Eddahibi, S., and Adnot, S. 2003. Role of VEGF-B in the lung during development of chronic hypoxic pulmonary hypertension. Am J Physiol Lung Cell Mol Physiol 284:L926-L937.
20. Rissanen, T. T., Markkanen, J. E., Gruchala, M., Heikura, T., Puranen, A., Kettunen, M. I., Kholova, I., Kauppinen, R. A., Achen, M. G., Stacker, S. A., et al. 2003. VEGF-D is the strongest angiogenic and lymphangiogenic effector among VEGFs delivered into skeletal muscle via adenoviruses. Circ Res 92:1098-1106.
21. Bhardwaj, S., Roy, H., Gruchala, M., Viita, H., Kholova, I., Kokina, I., Achen, M. G., Stacker, S. A., Hedman, M., Alitalo, K., et al. 2003. Angiogenic responses of vascular endothelial growth factors in periadventitial tissue. Hum Gene Ther 14:1451-1462.
22. Brkovic, A., and Sirois, M. G. 2007. Vascular permeability induced by VEGF family members in vivo: Role of endogenous PAF and NO synthesis. J Cell Biochem 100:727-737.
23. Abraham, D., Taghavi, S., Riml, P., Paulus, P., Hofmann, M., Baumann, C., Kocher, A., Klepetko, W., and Aharinejad, S. 2002. VEGF-A and -C but not -B mediate increased vascular permeability in preserved lung grafts. Transplantation 73:1703-1706.
24. Mould, A. W., Greco, S. A., Cahill, M. M., Tonks, I. D., Bellomo, D., Patterson, C., Zournazi, A., Nash, A., Scotney, P., Hayward, N. K., et al. 2005. Transgenic overexpression of vascular endothelial growth factor-B isoforms by endothelial cells potentiates postnatal vessel growth in vivo and in vitro. Circ Res 97:e60-70.
25. Silvestre, J. S., Tamarat, R., Ebrahimian, T. G., Le-Roux, A., Clergue, M., Emmanuel, F., Duriez, M., Schwartz, B., Branellec, D., and Levy, B. I. 2003. Vascular endothelial growth factor-B promotes in vivo angiogenesis. Circ Res 93:114-123.
26. Wright, C. E. 2002. Effects of vascular endothelial growth factor (VEGF)A and VEGFB gene transfer on vascular reserve in a conscious rabbit hindlimb ischaemia model. Clin Exp Pharmacol Physiol 29:1035-1039.
27. Tammela, T., Enholm, B., Alitalo, K., and Paavonen, K. 2005. The biology of vascular endothelial growth factors. Cardiovasc Res 65:550-563.
28. Wanstall, J. C., Gambino, A., Jeffery, T. K., Cahill, M. M., Bellomo, D., Hayward, N. K., and Kay, G. F. 2002. Vascular endothelial growth factor-B-deficient mice show impaired development of hypoxic pulmonary hypertension. Cardiovasc Res 55:361-368.
29. Puthalakath, H., Villunger, A., O'Reilly, L. A., Beaumont, J. G., Coultas, L., Cheney, R. E., Huang, D. C., and Strasser, A. 2001. Bmf: a proapoptotic BH3-only protein regulated by interaction with the myosin V actin motor complex, activated by anoikis. Science 293:1829-1832.
30. Okamura, S., Arakawa, H., Tanaka, T., Nakanishi, H., Ng, C. C., Taya, Y., Monden, M., and Nakamura, Y. 2001. p53DINP1, a p53-inducible gene, regulates p53-dependent apoptosis. Mol Cell 8:85-94.
31. Seidler, D. G., Goldoni, S., Agnew, C., Cardi, C., Thakur, M. L., Owens, R. T., McQuillan, D. J., and Iozzo, R. V. 2006. Decorin protein core inhibits in vivo cancer growth and metabolism by hindering epidermal growth factor receptor function and triggering apoptosis via caspase-3 activation. J Biol Chem 281:26408-26418.
32. Scotney, P. D., MacKenzie, A., Maccarone, P., Fabri, L. J., Scrofani, S. D., Gooley, P. R., and Nash, A. D. 2002. Human vascular endothelial growth factor B: characterization of recombinant isoforms and generation of neutralizing monoclonal antibodies. Clin Exp Pharmacol Physiol 29:1024-1029.
33. Sun, Y., Jin, K., Childs, J. T., Xie, L., Mao, X. O., and Greenberg, D. A. 2004. Increased severity of cerebral ischemic injury in vascular endothelial growth factor-B-deficient mice. J Cereb Blood Flow Metab 24:1146-1152.
34. Nishijima, K., Ng, Y. S., Zhong, L., Bradley, J., Schubert, W., Jo, N., Akita, J., Samuelsson, S. J., Robinson, G. S., Adamis, A. P., et al. 2007. Vascular endothelial growth factor-A is a survival factor for retinal neurons and a critical neuroprotectant during the adaptive response to ischemic injury. Am J Pathol 171:53-67.
35. Ferrara N, Carver-Moore K, Chen H, Dowd M, Lu L, O'shea Ks, Powell-Braxton L, Hillan Kj, and Moore Mw. 1996. Heterozygous embryonic lethality induced by targeted inactivation of the VEGF gene. Nature 380:439-442.
36. Karkkainen, M. J., Haiko, P., Sainio, K., Partanen, J., Taipale, J., Petrova, T. V., Jeltsch, M., Jackson, D. G., Talikka, M., Rauvala, H., et al. 2004. Vascular endothelial growth factor C is required for sprouting of the first lymphatic vessels from embryonic veins. Nat Immunol 5:74-80.
37. Dvorak, H. F., Brown, L. F., Detmar, M., and Dvorak, A. M. 1995. Vascular permeability factor/vascular endothelial growth factor, microvascular hyperpermeability, and angiogenesis. Am J Pathol 146:1029-1039.
38. Carmeliet, P., Moons, L., Luttun, A., Vincenti, V., Compernolle, V., De Mol, M., Wu, Y., Bono, F., Devy, L., Beck, H., et al. 2001. Synergism between vascular endothelial growth factor and placental growth factor contributes to angiogenesis and plasma extravasation in pathological conditions. Nat Med 7:575-583.
39. Joukov, V., Sorsa, T., Kumar, V., Jeltsch, M., Claesson-Welsh, L., Cao, Y., Saksela, O., Kalkkinen, N., and Alitalo, K. 1997. Proteolytic processing regulates receptor specificity and activity of VEGF-C. Embo J 16:3898-3911.
40. Shih, S. C., Ju, M., Liu, N., and Smith, L. E. 2003. Selective stimulation of VEGFR-1 prevents oxygen-induced retinal vascular degeneration in retinopathy of prematurity. J Clin Invest 112:50-57.
41. Plate, K. H., Beck, H., Danner, S., Allegrini, P. R., and Wiessner, C. 1999. Cell type specific upregulation of vascular endothelial growth factor in an MCA-occlusion model of cerebral infarct. J Neuropathol Exp Neurol 58:654-666.
42. Gerber, H. P., Malik, A. K., Solar, G. P., Sherman, D., Liang, X. H., Meng, G., Hong, K., Marsters, J. C., and Ferrara, N. 2002. VEGF regulates haematopoietic stem cell survival by an internal autocrine loop mechanism. Nature 417:954-958.
43. Shih, S. C., Ju, M., Liu, N., Mo, J. R., Ney, J. J., and Smith, L. E. 2003. Transforming growth factor beta1 induction of vascular endothelial growth factor receptor 1: mechanism of pericyte-induced vascular survival in vivo. Proc Natl Acad Sci USA 100:15859-15864.
44. Bates, R. C., Goldsmith, J. D., Bachelder, R. E., Brown, C., Shibuya, M., Oettgen, P., and Mercurio, A. M. 2003. Flt-1-dependent survival characterizes the epithelial-mesenchymal transition of colonic organoids. Curr Biol 13:1721-1727.
45. Gariano, R. F., Hu, D., and Helms, J. 2006. Expression of angiogenesis-related genes during retinal development. Gene Expr Patterns 6:187-192.
46. Beck, H., Acker, T., Puschel, A. W., Fujisawa, H., Carmeliet, P., and Plate, K. H. 2002. Cell type-specific expression of neuropilins in an MCA-occlusion model in mice suggests a potential role in post-ischemic brain remodeling. J Neuropathol Exp Neurol 61:339-350.
47. Moreno-Flores, M. T., Martin-Aparicio, E., Martin-Bermejo, M. J., Agudo, M., McMahon, S., Avila, J., Diaz-Nido, J., and Wandosell, F. 2003. Semaphorin 3C preserves survival and induces neuritogenesis of cerebellar granule neurons in culture. J Neurochem 87:879-890.
48. Seidel, M. F., Simard, J. M., Hunter, S. F., and Campbell, G. A. 1991. Isolation of arteriolar microvessels and culture of smooth muscle cells from cerebral cortex of guinea pig. Cell Tissue Res 265:579-587.
49. Nagai, N., Vanlinthout, I., and Collen, D. 1999. Comparative effects of tissue plasminogen activator, streptokinase, and staphylokinase on cerebral ischemic infarction and pulmonary clot lysis in hamster models. Circulation 100:2541-2546.
50. Darby, I. A., Bisucci, T., Desmouliere, A., and Hewitson, T. D. 2006. In situ hybridization using cRNA probes: isotopic and nonisotopic detection methods. Methods Mol Biol 326:17-31.
51. Li, X., Ponten, A., Aase, K., Karlsson, L., Abramsson, A., Uutela, M., Backstrom, G., Hellstrom, M., Bostrom, H., Li, H., et al. 2000. PDGF-C is a new protease-activated ligand for the PDGF alpha-receptor. Nat Cell Biol 2:302-309.
52. Sun, Y., Jin, K., Childs, J. T., Xie, L., Mao, X. O., and Greenberg, D. A. (2006) Vascular endothelial growth factor-B (VEGFB) stimulates neurogenesis: evidence from knockout mice and growth factor administration. Developmental biology 289, 329-335
53. Sun, Y., Jin, K., Childs, J. T., Xie, L., Mao, X. O. and Greenberg, D. A. (2004) Increased severity of cerebral ischemic injury in vascular endothelial growth factor-B-deficient mice. J. Cereb Blood Flow Metab 24, 1146-52.
54. Reichelt, M., Shi, S., Hayes, M., Kay, G., Batch, J., Gole, G. A. and Browning, J. (2003). Vascular endothelial growth factor-B and retinal vascular development in the mouse.
55. Li, X., Aase, K., Li, H., von Euler, G. and Eriksson, U. (2001). Isoform-specific expression of VEGF-B in normal tissues and tumors. Growth Factors 19, 49-59.
56. Li, X. and Eriksson, U. (2001). Novel VEGF family members: VEGF-B, VEGF-C and VEGF-D. Int J Biochem Cell Biol 33, 421-6.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for treating a degenerative disease in a mammalian subject, comprising
administering a VEGF-B polypeptide or functional variant or mimetic thereof in an amount effective to reduce or eliminate the degenerative disease in the mammalian subject without having any substantial angiogenic effect across all dosage levels.

2. The method of claim 1,
wherein the disease is a neurodegenerative disease, neurodegenerative ocular disease, neurovascular degenerative disease, glaucoma, diabetic retinopathy, retinitis pigmentosa, Usher syndrome, cone-rod dystrophies, Stargardt disease, choroideremia, retinoschisis, Leber amaurosis, retinoblastoma, muscular dystrophy, aging, autism, stroke, brain injury, or a combination thereof.

3. The method of claim 1,
wherein the disease is multiple sclerosis, Alzheimer's disease, amyotrophic lateral sclerosis (ALS), fibromyalgia, tinnitus, Parkinson's disease, Huntington's disease, or a combination thereof.

4. The method of claim 1,
wherein the disease is a condition involving apoptosis-mediated-cell death.

5. The method of claim 1,
wherein the disease is a condition associated with excessive apoptosis, AIDS, haematological disorders, aplastic anaemia, G6PD deficiency, myelodysplastic syndrome, T CD4+ lymphocytopenia, pathologies of tissue damage, myocardial infarction, ischemic renal damage, polycystic kidney disease, or a combination thereof.

6. The method of claim 1,
wherein the VEGF-B polypeptide functional variant or mimetic comprises a VEGF-B polypeptide fragment, peptide mimetic, nucleic acid, RNA, DNA, small chemical molecule, or antibody.

7. The method of claim 6,
wherein the VEGF-B polypeptide functional variant or mimetic comprises a conservative amino acid substitution or peptide mimetic substitution.

8. A method for inhibiting apoptosis in a cell or tissue of a mammalian subject, comprising
administering a VEGF-B polypeptide or functional variant or mimetic thereof in an amount effective to reduce or eliminate apoptosis in the cell or tissue of the mammalian subject without having any substantial angiogenic effect across all dosage levels.

9. The method of claim 8,
wherein the apoptosis occurs in a neural tissue.

10. The method of claim 8,
wherein the apoptosis occurs in endothelial cells, pericytes, Muller glial cells, neurons, nerves, cardiac myocytes, or myofibers.

11. The method of claim 9,
wherein the apoptosis occurs in brain or retina.

12. The method of claim 8,
wherein inhibition of apoptosis in the cell or tissue occurs without stimulating angiogenesis.

13. The method of claim 8,
wherein apoptosis in the cell or tissue is the result of neurodegenerative disease, neurodegenerative ocular disease, neurovascular degenerative disease, glaucoma, muscular dystrophy, aging, stroke, ischemia, diabetes, trauma, brain injury, or a combination thereof.

14. The method of claim 8,
wherein apoptosis in the cell or tissue is the result of multiple sclerosis, Alzheimer's disease, amyotrophic lateral sclerosis (ALS), fibromyalgia, tinnitus, Parkinson's disease, Huntington's disease, or a combination thereof.

15. The method of claim 8,
wherein the VEGF-B polypeptide functional variant or mimetic comprises a VEGF-B polypeptide fragment, peptide mimetic, nucleic acid, RNA, DNA, small chemical molecule, or antibody.

16. The method of claim 8,
wherein the VEGF-B polypeptide functional variant or mimetic comprises a conservative amino acid substitution or peptide mimetic substitution.

17. A method for identifying a compound which inhibits apoptosis in a cell or tissue without having any substantial angiogenic effect across all dosage levels, comprising:
contacting a VEGF-B polypeptide functional variant or mimetic test compound with a cell-based assay system comprising a cell expressing VEGFR-1 or neuropilin-1 and apoptosis/cell death related genes, and
detecting an effect of the test compound on VEGFR-1- or neuropilin-1-signaling and on inhibition of apoptosis/cell death related gene expression, effectiveness of the test compound in the assay being indicative of inhibition of apoptosis activity in the cell or tissue without having any substantial angiogenic effect across all dosage levels.

18. The method of claim 17, further comprising:
comparing the effect of the test compound to an effect of VEGF-B polypeptide on inhibition of apoptosis/cell death related gene expression.

19. The method of claim 17,
wherein the compound is a VEGF-B polypeptide functional variant or mimetic is a VEGF-B polypeptide fragment, VEGF-B peptide mimetic, small chemical molecule, nucleic acid, RNA, DNA, or antibody.

20. The method of claim 17,
wherein apoptosis/cell death related gene expression is BH3-only protein gene expression.

21. The method of claim 17,
wherein the BH3-only protein gene expression is expression of genes Bmf, Hrk, Puma, Noxa (Pmaip1), Bad, Bid, or Bik.

22. The method of claim 17,
wherein apoptosis/cell death related gene expression is expression of genes TrP53inp1 or DCN.
